(19) United States
(12) Patent Application Publication
(10) Pub. No.: US 2006/0128765 A1
(43) Pub. Date: Jun. 15, 2006

(54) 2-(Bicyclo)alkylamino-derivatives as mediators of chronic pain and inflammation

(76) Inventors: Michael R. Wood, Harleysville, PA (US); Scott D. Kuduk, Harleysville, PA (US); Mark G. Bock, Hatfield, PA (US); Ronald K. Chang, Wyncote, PA (US)

Correspondence Address: MERCK AND CO., INC, P O BOX 2000, RAHWAY, NJ, 07065-0907, US

(21) Appl. No.: 11/297,005

(22) Filed: Dec. 8, 2005

Related U.S. Application Data
(60) Provisional application No. 60/634,918, filed on Dec. 10, 2004.

(51) Int. Cl. A61K 31/44; A61K 31/195; A61K 31/165; A61K 31/16; C07D 213/56

(52) U.S. Cl. 514/357; 514/563; 514/613; 546/336; 562/450; 564/123

(57) ABSTRACT

Compounds disclosed herein are bradykinin B1 antagonist compounds useful in the treatment or prevention of symptoms such as pain and inflammation associated with the bradykinin B1 pathway.

### BACKGROUND OF THE INVENTION

This invention is directed to 2-(bicyclo)alkylamino derivatives as mediators of chronic pain and inflammation. In particular, this invention is directed to 2-(bicyclo)alkylamino derivatives that are bradykinin antagonists or inverse agonists.

Bradykinin (“BK”) is a kinin which plays an important role in the pathophysiological processes accompanying acute and chronic pain and inflammation. Bradykinin (BK), like other kinins, is an autacoid peptide produced by the catalytic action of kallikrein enzymes on plasma and tissue precursors termed kininogens. The biological actions of BK are mediated by at least two major G-protein-coupled BK receptors termed B1 and B2. It is generally believed that B2 receptors, but not B1 receptors, are expressed in normal tissues and that inflammation, tissue damage or bacterial infection can rapidly induce B1 receptor expression. This makes the B1 receptor a particularly attractive drug target. The putative role of kinins, and specifically BK, in the management of pain and inflammation has provided the impetus for developing potent and selective BK antagonists. In recent years, this effort has been heightened with the expectation that useful therapeutic agents with analgesic and anti-inflammatory properties would provide relief from maladies mediated through a BK receptor pathway (see e.g., M. G. Bock and J. Longmore, Current Opinion in Chem. Biol., 4:401-406(2000)). Accordingly, there is a need for novel compounds that are effective in blocking or reversing activation of bradykinin receptors. Such compounds would be useful in the management of pain and inflammation, as well as in the treatment or prevention of diseases and disorders mediated by bradykinin; further, such compounds are also useful as research tools (in vivo and in vitro).

### SUMMARY OF THE INVENTION

The present invention provides Compounds of Formula (Ia), (Ib) and (Ic) which are bradykinin antagonists or inverse agonists, pharmaceutical compositions containing such compounds, and methods of using them as therapeutic agents.

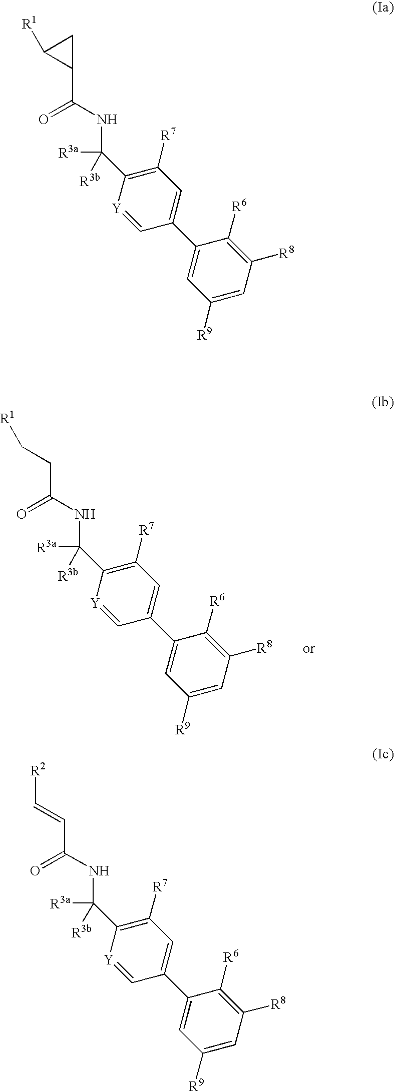

### DETAILED DESCRIPTION OF THE INVENTION

In one embodiment, the present invention provides compounds of Formula (Ia), (Ib) or (Ic): 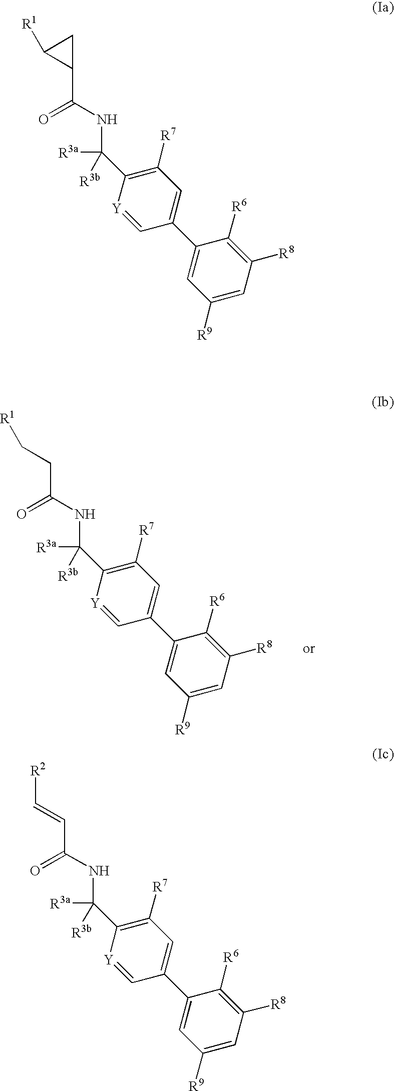
or a pharmaceutically acceptable salt thereof wherein
k is 0, 1, 2, 3, 4, or 5;
m is 2, 3, or 4;
n is 1, 2 or 3;
p is 1, 2, 3, 4, or 5;
Y is CH or N;
R¹ is selected from
(1) —(CH₂)ₖR²⁰,
(2) —(CH₂)ₖR³⁰,
(3) —(CH₂)ₖR⁴⁰,
(4) —(CH₂)ₖR⁵⁰, and
(5) —(CH₂)ₙC(O)R³²;
R² is selected from
(1) —(CH₂)ₚR²⁰,
(2) —(CH₂)ₚR³⁰,
(3) —(CH₂)ₚR⁴⁰,
(4) —(CH₂)ₚR⁵⁰, and
(5) —(CH₂)ₚC(O)R³²;
R^{3a} and R^{3b} are independently selected from
   (1) hydrogen, and
   (2) C₁₋₄ alkyl optionally substituted with 1 to 5 halogen atoms;
R⁶ is selected from
   (1) halogen,
   (2) CF₃,
   (3) CO₂R^{a},
   (4) C(O)NR^{b}R^{c},
   (5) OR^{a},
   (6) OSO₂R^{d}, and
   (7) optionally substituted heterocycle where the heterocycle is a 5-membered heteroaromatic ring having a ring heteroatom selected from N, O and S, and optionally having up to 3 additional ring nitrogen atoms, 4,5-dihydro-oxazolyl and 4,5-dihydro-1,2,4-oxadiazolyl, and wherein said substituent is 1 to 3 groups independently selected from C₁₋₄ alkyl optionally substituted with 1 to 5 halogen atoms, OR^{a} or OC(O)R^{a};
R⁷ is selected from
   (1) hydrogen and
   (2) halogen;
R⁸ and R⁹ are independently selected from
   (1) hydrogen,
   (2) halogen, and
   (3) C₁₋₄ alkyl optionally substituted with 1 to 5 halogen atoms;
R¹¹ is selected from
(1) hydrogen and
(2) (CH₂)ₖ—Ar optionally substituted with 1 to 3 groups independently selected from halogen, nitro, cyano, OR^{a}SR^{a}, CO₂R^{a}, C₁₋₄ alkyl and C₁₋₃ haloalkyl, wherein the Ar is selected from phenyl, pyridyl, 1,2-benzisothiazolyl, isoquinolinyl, 1,3-benzoxazolyl, quinazolinyl, 1,3-thiazolyl and 1,3,4-thiadiazolyl;
(3) C(O)OR^{a},
(4) C(O)—Ar1, wherein Ar1 is selected from indolyl and 1,2,4-triazolyl, and
(5) SO₂R^{d};
R¹² is 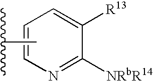
R¹³ and R¹⁴ are independently selected from
(1) hydrogen,
(2) C(O)OR^{a}, and
(3) C₁₋₄ alkyl optionally substituted with 1 to 5 groups independently selected from halogen, nitro, cyano and phenyl, or
R¹³ and R¹⁴ together form a bridging alkyl group of formula: (CH₂)ₘ;
R²⁰ is selected from
(1) OR^{a},
(2) —O-phenyl optionally substituted with with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, (CH₂)ₖR²¹, (CH₂)ₖN(R^{a})OR^{a} and 4,5-dihydro-1H-imidazolyl, and
(3) OC(O)NR^{b}R^{c};
R²¹ is selected from 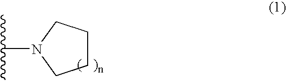
   optionally substituted with 1 to 2 groups, not on the same carbon, independently selected from C₁₋₄ alkyl, C₁₋₄ hydroxyalkyl, OR^{a}, CO₂R^{a}, R^{a}, (CH₂)ₙOR^{a}, phenyl, CH₂NR^{b}R^{c} and C(O)NR^{b}R^{c}; with the proviso that OR^{a} not be attached to a carbon attached to a nitrogen, 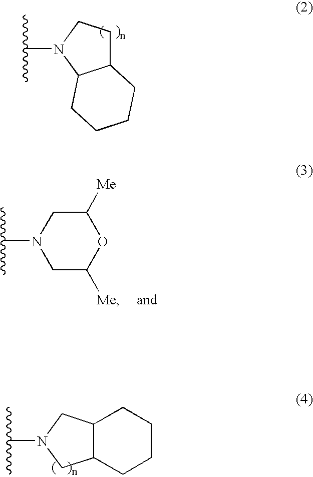
R³⁰ is selected from 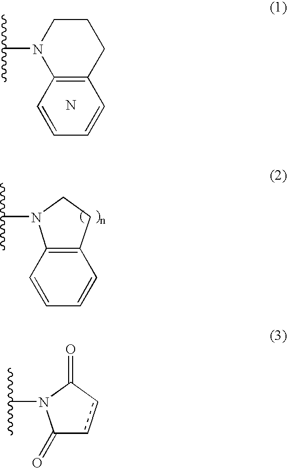
   optionally benzofused,
(4) indolyl,
(5) NR^{b}R^{c},
(6) NR^{b}C(O)OR^{a},
(7) NR^{b}SO₂R^{d},
(8) NR^{b}C(O)R^{a},
(9) NR^{b}C(O)-pyrimidinyl,
(10) NR^{b}(CH₂)ₙR³³,
(11) NR³¹(CH₂)ₖ-Ar2, wherein Ar2 is selected from phenyl,pyridyl and piperidinyl and Ar2 is optionally substituted with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl,
(12) NR^{b}(CH₂)ₖ-R¹²,
(13) N⁺(O—)R^{b}R^{c}, and
(14) a group selected from R³²;
R³¹ is selected from
(1) C(O)R^{a},
(2) R^{a}, and
(3) SO₂R^{d};
R³² is selected from

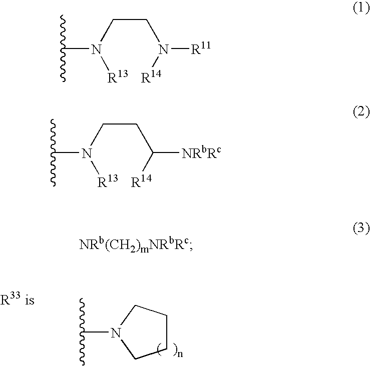

optionally substituted with 1 to 2 groups, not on the same carbon, independently selected from C₁₋₄ alkyl, C₁₋₄ haloalkyl, OR^{a}, CO₂R^{a}, R^{a}, (CH₂)ₙOR^{a}, phenyl, CH₂NR^{b}R^{c}; and C(O)NR^{b}R^{c}; with the proviso that OR^{a} not be attached to a carbon attached to a nitrogen;
R⁴⁰ is selected from
   (1) SO₂(OR^{a}),
   (2) SO₂R³²,
   (3) SO₂NR^{b}R^{c},
   (4) SO₂R^{d}, and
   (5) SR^{a};
R⁵⁰ is selected from
(1) a group from R¹²,
(2) C(O)OR^{a},
(3) Hydroxyl,
(4) —S(O)₂-C₁₋₄ alkyl,
(5) Ar3 is selected from from phenyl, pyridyl, piperidinyl, naphthyridinyl, imidazolyl, benzimidazolyl, indolyl, and thiophenyl and Ar3 is optionally substituted with with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl;
R^{a}is selected from
   (1) hydrogen,
   (2) C₁₋₄ alkyl optionally substituted with 1 to 5 halogen atoms,
   (3) (CH₂)ₖ-phenyl optionally substituted with 1 to 3 groups independently selected from halogen, cyano, nitro, OH, C₁₋₄ alkyloxy, C₃₋₆ cycloalkyl and C₁₋₄ alkyl optionally substituted with 1 to 5 halogen atoms,
   (4) C₃₋₆ cycloalkyl, and
   (5) pyridyl;
R^{b} and R^{c} are independently selected from
   (1) hydrogen,
   (2) C₁₋₄ alkyl optionally substituted with 1 to 5 groups independently selected from halogen, amino, mono-C₁₋₄alkylamino, di-C₁₋₄alkylamino, and SO₂R^{d},
   (3) (CH₂)ₖ-phenyl optionally substituted with 1 to 3 groups selected from halogen, cyano, nitro, OH, C₁₋₄ alkyloxy, C₃₋₆ cycloalkyl and C₁₋₄ alkyl optionally substituted with 1 to 5 halogen atoms, and
   (4) C₃₋₆ cycloalkyl, or
R^{b} and R^{c} together with the nitrogen atom to which they are attached form a 4-, 5-, or 6-membered aromatic or non-aromatic ring optionally containing an additional heteroatom selected from N, O, and S, wherein the S is optionally oxidized to the sulfone or sulfoxide; or
R^{b} and R^{c} together with the nitrogen atom to which they are attached form a cyclic imide;
R^{d} is selected from
   (1) C₁₋₄ alkyl optionally substituted with 1 to 5 halogen atoms,
   (2) C₁₋₄ alkyloxy,
   (3) phenyl optionally substituted with 1 to 3 groups selected from halogen, cyano, nitro, OH, C₁₋₄ alkyloxy, C₃₋₆ cycloalkyl and C₁₋₄ alkyl optionally substituted with 1 to 5 halogen atoms, and
   (4) pyridyl.

Within ths embodiment there is a genus of compounds wherein
R¹ is selected from
(1) —(CH₂)ₖR²⁰,
(2) —(CH₂)ₖR³⁰,
(3) —(CH₂)ₖR⁴⁰, and
(4) —(CH₂)ₙC(O)R³²; and
R² is selected from
(1) —(CH₂)ₚR²⁰,
(2) —(CH₂)ₚR⁴⁰,
(3) —(CH₂)ₚR⁵⁰, and
(4) —(CH₂)ₚC(O)R³².

Within this embodiment there is a genus of compounds wherein R^{3a} and R^{3b} are each independently selected from methyl and hydrogen.

Within ths embodiment there is a genus of compounds wherein
R⁶ is selected from
   (1) CF₃,
   (2) CO₂R^{a},
   (3) C(O)NR^{b}R^{c},
   (4) OR^{a}, and
   (5) optionally substituted heterocycle where the heterocycle is a 5-membered heteroaromatic ring having a ring heteroatom selected from N, O and S, and optionally having up to 3 additional ring nitrogen atoms, 4,5-dihydro-oxazolyl and 4,5-dihydro-1,2,4-oxadiazolyl, and wherein said substituent is 1 to 3 groups independently selected from C₁₋₄ alkyl optionally substituted with 1 to 5 halogen atoms, OR^{a} or OC(O)R^{a}.

Within ths genus there is a sub-genus of compounds wherein
R⁶ is selected from
   (1) CO₂R^{a},
   (2) OR^{a}, and
   (3) optionally substituted heterocycle where the heterocycle is a 5-membered heteroaromatic ring having a ring heteroatom selected from N, O and S, and optionally having up to 3 additional ring nitrogen atoms, 4,5-dihydro-oxazolyl and 4,5-dihydro-1,2,4-oxadiazolyl, and wherein said substituent is 1 to 3 groups independently selected from C₁₋₄ alkyl optionally substituted with 1 to 5 halogen atoms, OR^{a} or OC(O)R^{a}.

Within ths embodiment there is a genus of compounds wherein R⁷ is selected from fluorine and chlorine.

Within ths embodiment there is a genus of compounds wherein
R⁹ is selected from hydrogen, fluorine and chlorine, and
R⁸ is selected from fluorine and chlorine.

Within this embodiment there is a genus of compounds wherein
R¹¹ is selected from
(1) (CH₂)ₖ-Ar optionally substituted with 1 to 3 groups independently selected from halogen, nitro, cyano, OR^{a}SR^{a}, CO₂R^{a}, C₁₋₄ alkyl and C₁₋₃ haloalkyl, wherein the Ar is selected from phenyl, pyridyl, 1,2-benzisothiazolyl, isoquinolinyl, 1,3-benzoxazolyl, quinazolinyl, 1,3-thiazolyl and 1,3,4-thiadiazolyl;
(2) C(O)OR^{a}, and
(3) SO₂R^{d}.

Within this genus there is a sub-genus of compounds wherein
R¹¹ is
(CH₂)ₖ-Ar optionally substituted with 1 to 3 groups independently selected from halogen, OR^{a}, CO₂R^{a}, C₁₋₄ alkyl and C₁₋₃ haloalkyl, wherein the Ar is selected from phenyl and pyridyl.

Within this embodiment there is a genus of compounds wherein
R¹³ and R¹⁴ are independently selected from
(1) hydrogen, and
(2) C(O)OR^{a}, or
R¹³ and R¹⁴ together form a bridging alkyl group of formula: (CH₂)ₘ.

Within ths embodiment there is a genus of compounds wherein
R²⁰ is selected from
(1) OR^{a}, and
(2) —O-phenyl optionally substituted with with 1 to 3 groups independently selected from halogen, OR^{a}, C₁₋₄ alkyl, C₁₋₃ haloalkyl, (CH₂)ₖNR^{b}R^{c}; and (CH₂)ₖR²¹.

Within ths embodiment there is a genus of compounds wherein
R²¹ is 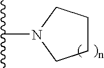
optionally substituted with 1 to 2 groups, not on the same carbon, independently selected from C₁₋₄ alkyl, C₁₋₄ hydroxyalkyl, OR^{a}, CO₂R^{a}, R^{a}, (CH₂)ₙOR^{a}, phenyl, CH₂NR^{b}R^{c}; and C(O)NR^{b}R^{c}; with the proviso that OR^{a} not be attached to a carbon attached to a nitrogen.

Within this genus there is a sub-genus of compounds wherein
R²¹ is selected from 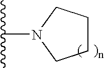
   optionally substituted with 1 to 2 groups, not on the same carbon, independently selected from C₁₋₄ alkyl, OR^{a}, phenyl and CH₂NR^{b}R^{c}; with the proviso that OR^{a} not be attached to a carbon attached to a nitrogen.

Within ths embodiment there is a genus of compounds wherein
R³⁰ is selected from 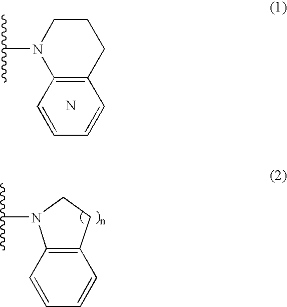
(3) NR^{b}R^{c},
(4) NR^{b}C(O)OR^{a},
(5) NR^{b}C(O)-pyrimidinyl,
(6) NR^{b}(CH₂)ₙR³³,
(7) NR³¹ (CH₂)ₖ-Ar2, wherein Ar2 is selected from phenyl,pyridyl and piperidinyl and Ar2 is optionally substituted with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl,
(8) NR^{b}(CH₂)ₖ-R¹²,
(9) a group selected from R³².

Within this genus there is a sub-genus of compounds wherein
R³⁰ is selected from 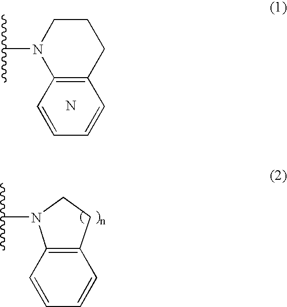
(3) NR^{b}R^{c},
(4) NR^{b}(CH₂)ₙR³³,
(5) NR³¹(CH₂)ₖ-Ar2, wherein Ar2 is selected from phenyl and pyridyland Ar2 is optionally substituted with 1 to 3 groups independently selected from halogen, OR^{a}, C₁₋₄ alkyl, C₁₋₃ haloalkyl, and (CH₂)ₖNR^{b}R^{c}, and
(6) NR^{b}(CH₂)ₖ-R¹².

Within ths embodiment there is a genus of compounds wherein
R³¹ is
R^{a}.

Within this genus there is a sub-genus of compounds wherein
R³¹ is Hydrogen.

Within this embodiment there is a genus of compounds wherein
R³³ is 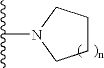
   optionally substituted with 1 to 2 groups, not on the same carbon, independently selected from C₁₋₄ alkyl, OR^{a}, phenyl, and CH₂NR^{b}R^{c}; with the proviso that OR^{a} not be attached to a carbon attached to a nitrogen.

Within ths embodiment there is a genus of compounds wherein
R⁴⁰ is selected from
   (1) SO₂(OR^{a}), and
   (2) SO₂R³².

Within this genus there is a sub-genus of compounds wherein
R⁴⁰ is
   SO₂R³².

Within ths embodiment there is a genus of compounds wherein
R⁵⁰ is selected from
(1) a group from R¹²,
(2) Hydroxyl, and
(3) Ar3 is selected from from phenyl, pyridyl, piperidinyl, naphthyridinyl, imidazolyl, benzimidazolyl, indolyl, and thiophenyl and Ar3 is optionally substituted with with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl.

Within this genus there is a sub-genus of compounds wherein
R⁵⁰ is selected from
(1) a group from R¹²,
(2) Hydroxyl, and
(3) Ar3 is selected from from phenyl, pyridyl, piperidinyl, naphthyridinyl, imidazolyl, benzimidazolyl, indolyl, and thiophenyl and Ar3 is optionally substituted with with 1 to 3 groups independently selected from halogen, OR^{a}, C₁₋₄ alkyl, C₁₋₃ haloalkyl, and (CH₂)ₖNR^{b}R^{c}.

Within ths embodiment there is a genus of compounds wherein
k is 0, 1, 2, 3 or 4; and
p is 0, 1, 2, 3 or 4.

Within this genus there is a sub-genus of cokpounds wherein
k is 0, 1, 2 or 3; and
p is 1, 2 or 3.

Within ths embodiment there is a genus of compounds wherein
m is 3 or 4.

Within this genus there is a sub-genus of compounds wherein
m is 3.

Within ths embodiment there is a genus of compounds wherein
n is 1 or 2.

Within ths embodiment there is a genus of compounds wherein
R¹ is selected from
(1) —(CH₂)ₖR²⁰,
(2) —(CH₂)ₖR³⁰,
(3) —(CH₂)ₖR⁴⁰, and
(4) —(CH₂)ₙC(O)R³²;
R² is selected from
(1) —(CH₂)ₚR²⁰,
(2) —(CH₂)ₚR⁴⁰,
(3) —(CH₂)ₚR⁵⁰, and
(4) —(CH₂)ₚC(O)R³²;
R^{3a} and R^{3b} are each independently selected from methyl and hydrogen;
R⁶ is selected from
   (1) CF₃,
   (2) CO₂R^{a},
   (3) C(O)NR^{b}R^{c},
   (4) OR^{a}, and
   (5) optionally substituted heterocycle where the heterocycle is a 5-membered heteroaromatic ring having a ring heteroatom selected from N, O and S, and optionally having up to 3 additional ring nitrogen atoms, 4,5-dihydro-oxazolyl and 4,5-dihydro-1,2,4-oxadiazolyl, and wherein said substituent is 1 to 3 groups independently selected from C₁₋₄ alkyl optionally substituted with 1 to 5 halogen atoms, OR^{a} or OC(O)R^{a};
R⁷ is selected from fluorine and chlorine;
R⁹ is selected from hydrogen, fluorine and chlorine;
R⁸ is selected from fluorine and chlorine;
R¹¹ is selected from
(1) (CH₂)ₖ-Ar optionally substituted with 1 to 3 groups independently selected from halogen, nitro, cyano, OR^{a}SR^{a}, CO₂R^{a}, C₁₋₄ alkyl and C₁₋₃ haloalkyl, wherein the Ar is selected from phenyl, pyridyl, 1,2-benzisothiazolyl, isoquinolinyl, 1,3-benzoxazolyl, quinazolinyl, 1,3-thiazolyl and 1,3,4-thiadiazolyl;
(2) C(O)OR^{a}, and
(3) SO₂R^{d};
R¹³ and R¹⁴ are independently selected from
(1) hydrogen, and
(2) C(O)OR^{a}, or
R¹³ and R¹⁴ together form a bridging alkyl group of formula: (CH₂)ₘ;.
R²⁰ is selected from
(1) OR^{a}, and
(2) —O-phenyl optionally substituted with with 1 to 3 groups independently selected from halogen, OR^{a}, C₁₋₄ alkyl, C₁₋₃ haloalkyl, (CH₂)ₖNR^{b}R^{c}; and (CH₂)ₖR²¹;
R²¹ is 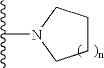
   optionally substituted with 1 to 2 groups, not on the same carbon, independently selected from C₁₋₄ alkyl, C₁₋₄ hydroxyalkyl, OR^{a}, CO₂R^{a}, R^{a}, (CH₂)ₙOR^{a}, phenyl, CH₂NR^{b}R^{c}; and C(O)NR^{b}R^{c}; with the proviso that OR^{a} not be attached to a carbon attached to a nitrogen;
R³⁰ is selected from 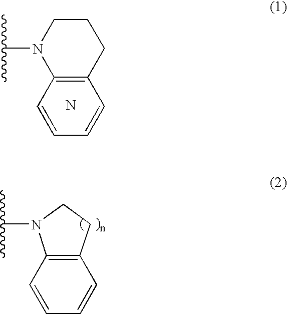
(3) NR^{b}R^{c},
(4) NR^{b}C(O)OR^{a},
(5) NR^{b}C(O)-pyrimidinyl,
(6) NR^{b}(CH₂)ₙR³³,
(7) NR³¹(CH₂)ₖ-Ar2, wherein Ar2 is selected from phenyl,pyridyl and piperidinyl and Ar2 is optionally substituted with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl,
(8) NR^{b}(CH₂)ₖ-R¹²,
(9) a group selected from R³²;
R³¹ is
R^{a};
R³³ is 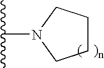
   optionally substituted with 1 to 2 groups, not on the same carbon, independently selected from C₁₋₄ alkyl, OR^{a}, phenyl, and CH₂NR^{b}R^{c}; with the proviso that OR^{a} not be attached to a carbon attached to a nitrogen;
R⁴⁰ is selected from
   (1) SO₂(OR^{a}), and
   (2) SO₂R³²;
R⁵⁰ is selected from
(1) a group from R¹²,
(2) Hydroxyl, and
(3) Ar3 is selected from from phenyl, pyridyl, piperidinyl, naphthyridinyl, imidazolyl, benzimidazolyl, indolyl, and thiophenyl and Ar3 is optionally substituted with with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl.
k is 0, 1, 2, 3 or 4;
p is 0, 1, 2, 3 or 4;
m is 3 or 4; and
n is 1 or 2.

Within this genus there is a class of compounds wherein
R⁶ is selected from
   (1) CO₂R^{a},
   (2) OR^{a}, and
   (3) optionally substituted heterocycle where the heterocycle is a 5-membered heteroaromatic ring having a ring heteroatom selected from N, O and S, and optionally having up to 3 additional ring nitrogen atoms, 4,5-dihydro-oxazolyl and 4,5-dihydro-1,2,4-oxadiazolyl, and wherein said substituent is 1 to 3 groups independently selected from C₁₋₄ alkyl optionally substituted with 1 to 5 halogen atoms, OR^{a} or OC(O)R^{a};
R¹¹ is
(CH₂)ₖ-Ar optionally substituted with 1 to 3 groups independently selected from halogen, OR^{a}, CO₂R^{a}, C₁₋₄ alkyl and C₁₋₃ haloalkyl, wherein the Ar is selected from phenyl and pyridyl;
R²¹ is selected from 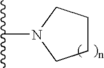
   optionally substituted with 1 to 2 groups, not on the same carbon, independently selected from C₁₋₄ alkyl, OR^{a}, phenyl and CH₂NR^{b}R^{c}; with the proviso that OR^{a} not be attached to a carbon attached to a nitrogen;
R³⁰ is selected from 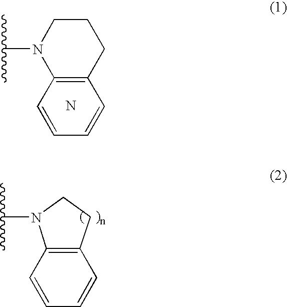
(3) NR^{b}R^{c},
(4) NR^{b}(CH₂)ₙR³³,
(5) NR³¹ (CH₂)ₖ-Ar2, wherein Ar2 is selected from phenyl and pyridyland Ar2 is optionally substituted with 1 to 3 groups independently selected from halogen, OR^{a}, C₁₋₄ alkyl, C₁₋₃ haloalkyl, and (CH₂)ₖNR^{b}R^{c}, and
(6) NR^{b}(CH₂)ₖ-R¹²;
R³¹ is Hydrogen;
R⁴⁰ is
   SO₂R³²;
R⁵⁰ is selected from
(1) a group from R²,
(2) Hydroxyl, and
(3) Ar3 is selected from from phenyl, pyridyl, piperidinyl, naphthyridinyl, imidazolyl, benzimidazolyl, indolyl, and thiophenyl and Ar3 is optionally substituted with with 1 to 3 groups independently selected from halogen, OR^{a}, C₁₋₄ alkyl, C₁₋₃ haloalkyl, and (CH₂)ₖNR^{b}R^{c};
k is 0, 1, 2 or 3;
p is 1, 2 or 3; and
m is 3.

Illustrating the invention are the following compounds:
Methyl 3,3′-difluoro-4′-{[(3-hydroxybenzoyl)amino]methyl}biphenyl-2-carboxylate,
Methyl 3,3′-difluoro-4′-[({4-[(4-pyridin-4-ylpiperazin-1-yl)sulfonyl]benzoyl}amino)methyl]biphenyl-2-carboxylate,
Methyl 3,3′-difluoro-4′-{[(4-{[(2-piperidin-1-ylethyl)amino]sulfonyl}benzoyl)amino]methyl}biphenyl-2-carboxylate,
Methyl 3,3′-difluoro-4′-[({4-[(4-pyridin-4-ylpiperazin-1-yl)carbonyl]benzoyl}amino)methyl]biphenyl-2-carboxylate,
Methyl 4′-({[3-(aminomethyl)benzoyl]amino}methyl)-3,3′-difluorobiphenyl-2-carboxylate,
Methyl 3,3′-difluoro-4′-{[(3-{[(trifluoroacetyl)amino]methyl}benzoyl)amino]methyl}biphenyl-2-carboxylate,
Methyl 3,3′-difluoro-4′-{[(3-{[(3-piperidin-1-ylpropanoyl)amino]methyl}benzoyl)amino]methyl}biphenyl-2-carboxylate,
Methyl 3,3′-difluoro-4′-{[(3-{[(2-piperidin-1-ylethyl)amino]sulfonyl}benzoyl)amino]methyl}biphenyl-2-carboxylate,
Methyl 3,3′-difluoro-4′-[({3-[(pyrimidin-5-ylcarbonyl)amino]benzoyl}amino)methyl]biphenyl-2-carboxylate, and
Methyl 3,3′-difluoro-4′-({[2-(4-pyridin-4-ylpiperazin-1-yl)isonicotinoyl]amino}methyl)biphenyl-2-carboxylate.

Unless otherwise stated, the following terms have the meanings indicated below:

“Alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec-and tert-butyl, pentyl, hexyl, beptyl and the like.

“Alkenyl” means a linear or branched carbon chain containing at least one C═C bond. Examples of alkenyl include allyl, 2-butenyl, 3-butenyl, 1-methyl-2-propenyl, and the like.

“Aryl” means phenyl or naphthyl.

“Halogen” means fluorine, chlorine, bromine and iodine.

“Optionally substituted” is intended to include both substituted and unsubstituted. Thus, for example, optionally substituted aryl could represent a pentafluorophenyl or a phenyl ring.

Applicants point out that the floating “N” in the center of the ring system in the group: 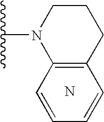
indicates that the “N” may reside at any available position of the aromatic ring.

Applicants also point out that each occurance of a variable may be independently selected from the list of choices defining said variable.

Optical Isomers—Diastereomers—Geometric Isomers—Tautomers

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The above Formula (Ia), (Ib) or (Ic) is shown without a definitive stereochemistry at certain positions. The present invention includes all stereoisomers of Formula (Ia), (Ib) or (Ic) and pharmnaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general Formula (Ia), (Ib) or (Ic) may be obtained by stereospecific synthesis using optically pure starting materials or reagents of known configuration.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form known as keto-enol tautomers. The individual tautomers as well as mixture thereof are encompassed with compounds of Formula (Ia), (Ib) or (Ic).

Salts

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Preferred are the ammonium, calcium, magnesium, potassium and sodium salts. Salts prepared from pharmaceutically acceptable organic non-toxic bases include salts of primary, secondary, and tertiary amines derived from both naturally occurring and synthetic sources. Pharmaceutically acceptable organic non-toxic bases from which salts can be formed include, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

Prodrugs

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

Pharmaceutical Compositions

Another aspect of the present invention provides pharmaceutical compositions which comprises a compound of Formula (Ia), (Ib) or (Ic) and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula (Ia), (Ib) or (Ic), additional active ingredient(s), and pharmaceutically acceptable excipients.

The pharmaceutical compositions of the present invention comprise a compound represented by Formula (Ia), (Ib) or (Ic) (or pharmaceutically acceptable salts thereof) as an active ingredient, a pharmaceutically acceptable carrier and optionally other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula (Ia), (Ib) or (Ic) or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula (Ia), (Ib) or (Ic) or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula (Ia), (Ib) or (Ic). The compounds of Formula (Ia), (Ib) or (Ic), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula (Ia), (Ib) or (Ic) of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula (Ia), (Ib) or (Ic), or pharmaceutically acceptable salts thereof, may also be prepared in powder or liquid concentrate form.

The following are examples of representative pharmaceutical dosage forms for the compounds of Formula (Ia), (Ib) or (Ic):

| Injectable Suspension (I.M.) | mg/mL |
|---|---|
| Compound of Formula (Ia), (Ib) or (Ic) | 10 |
| Methylcellulose | 5.0 |
| Tween 80 | 0.5 |
| Benzyl alcohol | 9.0 |
| Benzalkonium chloride | 1.0 |
| Water for injection to a total volume of 1 mL | |

| Tablet | mg/tablet |
|---|---|
| Compound of Formula (Ia), (Ib) or (Ic) | 25 |
| Microcrystalline Cellulose | 415 |
| Povidone | 14.0 |
| Pregelatinized Starch | 43.5 |
| Magnesium Stearate | 2.5 |
| | 500 |

| Capsule | mg/capsule |
|---|---|
| Compound of Formula (Ia), (Ib) or (Ic) | 25 |
| Lactose Powder | 573.5 |
| Magnesium Stearate | 1.5 |
| | 600 |

Utilities

Compounds of this invention are antagonists or inverse agonists of bradykinin receptor, in particular the bradykinin B1 receptor, and as such are useful in the treatment and prevention of diseases and conditions mediated through the bradykinin receptor pathway such as pain and inflammation. The compounds would be effective in the treatment or prevention of pain including, for example, visceral pain (such as pancreatitis, interstitial cystitis, renal colic, prostatitis, chronic pelvic pain), neuropathic pain (such as postherpetic neuralgia, acute zoster pain, nerve injury, the “dynias”, e.g., vulvodynia, phantom limb pain, root avulsions, radiculopathy, painful traumatic mononeuropathy, painful entrapment neuropathy, carpal tunnel syndrome, ulnar neuropathy, tarsal tunnel syndrome, painful diabetic neuropathy, painful polyneuropathy, trigeminal neuralgia), central pain syndromes (potentially caused by virtually any lesion at any level of the nervous system including but not limited to stroke, multiple sclerosis, spinal cord injury), and postsurgical pain syndromes (eg, postmastectomy syndrome, postthoracotomy syndrome, stump pain)), bone and joint pain (osteoarthritis), spine pain (e.g., acute and chronic low back pain, neck pain, spinal stenosis), shoulder pain, repetitive motion pain, dental pain, sore throat, cancer pain, burn pain, myofascial pain (muscular injury, fibromyalgia), postoperative, perioperative pain and preemptive analgesia (including but not limited to general surgery, orthopedic, and gynecological), chronic pain, dysmenorrhea (primary and secodnary), as well as pain associated with angina, and inflammatory pain of varied origins (e.g. osteoarthritis, rheumatoid arthritis, rheumatic disease, teno-synovitis and gout, ankylosing spondylitis, bursitis).

Further, the compounds of this invention can also be used to treat hyperreactive airways and to treat inflammatory events associated with airways disease e.g. asthma including allergic asthma (atopic or non-atopic) as well as exercise-induced bronchoconstriction, occupational asthma, viral- or bacterial exacerbation of asthma, other non-allergic asthmas and “wheezy-infant syndrome”. Compounds of the present invention may also be used to treat chronic obstructive pulmonary disease including emphysema, adult respiratory distress syndrome, bronchitis, pneumonia, allergic rhinitis (seasonal and perennial), and vasomotor rhinitis. They may also be effective against pneumoconiosis, including aluminosis, anthracosis, asbestosis, chalicosis, ptilosis, siderosis, silicosis, tabacosis and byssinosis.

Compounds of the present invention may also be used for the treatment of inflammatory bowel disease including Crohn's disease and ulcerative colitis, irritable bowel syndrome, pancreatitis, nephritis, cystitis (interstitial cystitis), uveitis, inflammatory skin disorders such as psoriasis and eczema, rheumatoid arthritis and edema resulting from trauma associated with bums, sprains or fracture, cerebral edema and angioedema (including hereditary angioedema and drug-induced angioedema such as that caused by angiotensin converting enzyme (ACE) or ACE/neutral endopeptidase inhibitors, e.g. omepatrilat). They may be used to treat diabetic vasculopathy, diabetic neuropathy, diabetic retinopathy, post capillary resistance or diabetic symptoms associated with insulitis (e.g. hyperglycemia, diuresis, proteinuria and increased nitrite and kallikrein urinary excretion). They may be used as smooth muscle relaxants for the treatment of spasm of the gastrointestinal tract or uterus. Additionally, they may be effective against liver disease, multiple sclerosis, cardiovascular disease, e.g. atherosclerosis, congestive heart failure, myocardial infarct; neurodegenerative diseases, eg. Parkinson's and Alzheimers disease, epilepsy, septic shock e.g. as anti-hypovolemic and/or anti-hypotensive agents, headache including cluster headache, migraine including prophylactic and acute use, stroke, closed head trauma, cancer, sepsis, gingivitis, osteoporosis, benign prostatic hyperplasia and hyperactive bladder. Animal models of these diseases and conditions are generally well known in the art, and may be suitable for evaluating compounds of the present invention for their potential utilities. Finally, compounds of the present invention are also useful as research tools (in vivo and in vitro).

The compounds of this invention are useful in the treatment of pain and inflammation by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

The compounds would be effective in the treatment or prevention of pain including, for example, bone and joint pain (osteoarthritis), repetitive motion pain, dental pain, cancer pain, myofascial pain (muscular injury, fibromyalgia), perioperative pain (general surgery, oral surgery, gynecological), neuropathic pain (post-herpetic neuralgia), and chronic pain by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three, times a week.

In particular, inflammatory pain such as, for example, inflammatory airways disease (chronic obstructive pulmonary disease) would be effectively treated by the compounds of this invention by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Further, the compounds of this invention can additionally be used to treat asthma, inflammatory bowel disease, rhinitis, pancreatitis, cystitis (interstitial cystitis), uveitis, inflammatory skin disorders, rheumatoid arthritis and edema resulting from trauma associated with burns, sprains or fracture by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used subsequent to surgical intervention (e.g. as post-operative analgesics) and to treat inflammatory pain of varied origins (e.g. osteoarthritis, rheumatoid arthritis, rheumatic disease, teno-synovitis and gout) as well as for the treatment of pain associated with angina, menstruation or cancer by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat diabetic vasculopathy, post capillary resistance or diabetic symptoms associated with insulitis (e.g. hyperglycemia, diuresis, proteinuria and increased nitrite and kallikrein urinary excretion) by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat inflammatory skin disorders such as psoriasis and eczema by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used as smooth muscle relaxants for the treatment of spasm of the gastrointestinal tract or uterus or in the therapy of Crohn's disease, ulcerative colitis or pancreatitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Such compounds may be used therapeutically to treat hyperreactive airways and to treat inflammatory events associated with airways disease e.g. asthma, and to control, restrict or reverse airways hyperreactivity in asthma by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may be used to treat intrinsic and extrinsic asthma including allergic asthma (atopic or non-atopic) as well as exercise-induced bronchoconstriction, occupational asthma, viral or bacterial exacerbated asthma, other non-allergic asthmas and “wheezy-infant syndrome” by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

They may also be effective against pneumoconiosis, including aluminosis, anthracosis, asbestosis, chalicosis, ptilosis, siderosis, silicosis, tabacosis and byssinosis was well as adult respiratory distress syndrome, chronic obstructive pulmonary or airways disease, bronchitis, allergic rhinitis, and vasomotor rhinitis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Additionally, they may be effective against liver disease, multiple sclerosis, atherosclerosis, Alzheimer's disease, septic shock e.g. as anti-hypovolemic and/or anti-hypotensive agents, cerebral edema, headache including cluster headache, migraine including prophylactic and acute use, closed head trauma, irritable bowel syndrome and nephritis by the administration of a tablet, cachet, or capsule each containing, for example, 0.1 mg, 0.5 mg, 1 mg, 3 mg, 5 mg, 10 mg, 25 mg, 50 mg, 100 mg, 125 mg, 250 mg, or 500 mg of a compound of this invention once every three to four hours, once, twice or three times a day, or (in an extended release formulation) once, twice or three times a week.

Combination Therapy

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (1) morphine and other opiate receptor agonists including codeine, oxycodone, propoxyphene (Darvon) and tramadol; (2) non-steroidal antiinflammatory drugs (NSAIDs) including COX-2 inhibitors such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone), and the coxibs (celecoxib, valecoxib, rofecoxib and etoricoxib); (3) corticosteroids such as betamethasone, budesonide, cortisone, dexamethasone, hydrocortisone, methylprednisolone, prednisolone, prednisone and triamcinolone; (4) histamine H1 receptor antagonists such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, desloratadine, fexofenadine and levocetirizine; (5) histamine H2 receptor antagonists such as cimetidine, famotidine and ranitidine; (6) proton pump inhibitors such as omeprazole, pantoprazole and esomeprazole; (7) leukotriene antagonists and 5-lipoxygenase inhibitors such as zafirlukast, montelukast, pranlukast and zileuton; (8) drugs used for angina, myocardial ischemia including nitrates such as nitroglycerin and isosorbide nitrates, beta blockers such as atenolol, metoprolol, propranolol, acebutolol ,betaxolol, bisoprolol, carteolol, labetalol, nadolol, oxprenolol, penbutolol, pindolol, sotalol and timolol, and calcium channel blockers such as diltiazam, verapamil, nifedipine, bepridil, felodipine, flunarizine, isradipine, nicardipine and nimodipine; (9) incontinence medications such as antimuscarinics, e.g., tolterodine and oxybutinin); (10) gastrointestinal antispasmodics (such as atropine, scopolamine, dicyclomine, antimuscarinics, as well as diphenoxylate); skeletal muscle relaxants (cyclobenzaprine, carisoprodol, chlorphenesin, chlorzoxazone, metaxalone, methocarbamol, baclofen, dantrolene, diazepam, or orphenadrine); (11) gout medications such as allopurinol, probenicid and colchicine; (12) drugs for rheumatoid arthritis such as methotrexate, auranofin, aurothioglucose and gold sodium thiomalate; (13) drugs for osteoporosis such as alendronate and raloxifene; (14) decongestants such as pseudoephedrine and phenylpropanolamine; (15) local anesthetics; (16) anti-herpes drugs such as acyclovir, valacyclovir and famcyclovir; (17) anti-emetics such as ondansetron and granisetron; (18) migraine drugs such as the triptans (e.g. rizatriptan, sumatriptan), ergotamine, dihydroergotamine, CGRP antagonists, (19) antidepressants (e.g., tricyclic antidepressants (such as doxepin, clomipramine, imipramine, amitriptyline, maprotiline, nortriptyline), serotonin-selective/serotonin and norepinephrine reuptake inhibitors (such as paroxetine, fluoxetine, duloxetine, vanlafexine), beta-adrenergic blockers; (20) VR1 antagonsits; (21) anticonvulsants (e.g., gabapentin, pregabalin, lamotrigine, topiramate, carbamazepine, oxcarbazepine, phenytoin); (22) glutamate antagonists (e.g., ketamine and other NMDA antagonists, NR2B antagonists); (23) acetaminophen; (24) CCR2 antagonists; (25) PDE4 antagonists such as roflumilast; (26) tegaserod; (27) alosetron; (28) topiramate; (29) cathepsin K inhibitors; and (30) ACE/NEP inhibitors such as omepatrilat.

Biological Evaluation

Assessing the Affinity of Selected Compounds to Bind to the Bradykinin B1 or B2 Receptor

Radioligand binding assays are performed using membranes from CHO cells that stably express the human, rabbit, rat, or dog B1 receptors or CHO cells that express the human B2 receptor. For all receptor types, cells are harvested from culture flasks in PBS/1 mM EDTA and centrifuged at 1000×g for 10 minutes. The cell pellets are homogenized with a polytron in ice cold 20 mM HEPES, 1 mM EDTA, pH 7.4 (lysis buffer) and centrifuged at 20,000×g for 20 minutes. The membrane pellets are rehomogenized in lysis buffer, centrifuged again at 20,000×g and the final pellets are resuspended at 5 mg protein/ml in assay buffer (120 mM NaCl, 5 mM KCl, 20 mM REPES, pH 7.4) supplemented with 1% BSA and frozen at −80° C.

On the day of assay, membranes are centrifuged at 14,000×g for 5 minutes and resuspended to the desired protein concentration in assay buffer containing lOOnM enaliprilat, 140 μg/mL bacitracin and 0.1% BSA. 3H-des-arglo, leu9 kallidin is the radioligand used for the human and rabbit B1 receptors, 3H-des-arg10 kallidin is used for the rat and dog B1 receptors, and 3H-bradykinin is used to label the human B2 receptor.

For all assays, compounds are diluted from DMSO stock solutions with 4 μL added to assay tubes for a final DMSO concentration of 2%. This is followed by the addition of 100 μL radioligand and 100 μL of the membrane suspension. Nonspecific binding for the B1 receptor binding assays is determined using 1 μM des-arglo kallidin and nonspecific binding for the B2 receptor is determined with 1 μM bradykinin. Tubes are incubated at room temperature (22° C.) for 60 minutes followed by filtration using a Tomtec 96-well harvesting system. Radioactivity retained by the filter is counted using a Wallac Beta-plate scintillation counter.

The compounds of this invention have affinity for the B1 receptor in the above assay as demonstrated by results of less than 5 μM. It is advantageous that the assay results be less than 1 μM, even more advantageous for the results be less than 0.5 μM. It is further advantageous that compounds of this invention have affinity for the bradykinin B1 receptor over the bradykinin B2 receptor; more advantageously, the affinity for the B1 receptor is at least 10 fold, and preferably over 100 fold, over that for the B2 receptor.

Assay for Bradykinin B1 Antagonists

B1 agonist-induced calcium mobilization was monitored using a Fluorescence Imaging Plate Reader (FLIPR). CHO cells expressing the B1 receptor were plated in 96 or 384 well plates and allowed to incubate in Iscove's modified DMEM overnight. Wells were washed two times with a physiological buffered salt solution and then incubated with 4 uM Fluo-3 for one hour at 37° C. The plates were then washed two times with buffered salt solution and 100 uL of buffer was added to each well. Plates were placed in the FLIPR unit and allowed to equilibrate for two minutes. The test compound was then added in 50 ul volumes followed five minutes later by 50 ul of agonist (des-arglo kallidin). Relative fluorescence peak heights in the absence and presence of antagonist were used to calculate the degree of inhibition of the B1 receptor agonist response by the test compound. Eight to ten concentrations of test compound were typically evaluated to construct an inhibition curve and determine IC50 values using a four-parameter nonlinear regression curve fitting routine.

Assay for Bradykinin Inverse Agonists

Inverse agonist activity at the human B1 receptor was evaluated using transiently transfected HEK293 cells. One day following transfection cell flasks were labeled overnight with 6 uCi/ml [³H]myo-inositol. On the day of assay, the media was removed and the attached cells were gently rinsed with 2×20 ml of phosphate-buffered saline. Assay buffer (HEPES buffered physiological salts, pH 7.4) was added and the cells were detached by tapping of the flask. The cells were centrifuged at 800×g for five minutes and resuspended at 1×10⁶ cells/ml in assay buffer supplemented with 10 mM lithium chloride. After 10 minutes at room temperature, one-half ml aliquots were distributed to tubes containing test compound or vehicle. After an additional 10 minutes the tubes were transferred to a 37° C. water bath for 30 minutes. The incubation was terminated by the addition of a 12% perchloric acid solution and the tubes were placed on ice for 30 minutes. The acid was then neutralized with KOH and the tubes centrifuged to pellet precipitated material. [³H]Inositol monophosphate formed was recovered by standard ion exchange chromatographic techniques and quantitated by liquid scintillation counting. Inverse agonist activity was determined by the degree to which a test compound reduced basal (cells incubated with vehicle) levels of [³H]inositol monophosphate accumulation.

Abbreviations Used

AIBN 2,2′-azobisisobutyronitrile
Bu Butyl
DMF Dimethylformamide
DMSO Dimethyl dimethyl sulfoxide
EDC or EDCI 1-(3-dimethylaminopropyl)3-ethylcarbodiimide HCl
ES (or ESI)—MS electron spray ionization—mass spectroscopy
EtOAc ethyl acetate
HBT or HOBt 1-hydroxybenzotriazole hydrate
HPLC high pressure liquid chromatography
Me Methyl
MeOH Methanol
NBS N-bromosuccinimde
NMR nuclear magnetic resonance
Ph Phenyl
rt room temperature
TEA Triethylamine
Tf triflate (trifluoromethanesulfonyl)
TFA trifluoroacetic acid
THF Tetrahydrofuran

The compounds of the present invention can be prepared according to the following reaction schemes and examples, or modifications thereof, using readily available starting materials, reagents, and conventional synthesis procedures. In these reactions, it is also possible to make use of variants which are themselves known to those of ordinary skill in this art, but are not mentioned in greater detail.

Applicants point out that for purposes of the Schemes, the designation “X” may be either “—CH₂—” or “H,H”. Thus, for example, when “X” is “—CH₂—” the group of which it is a part is a cyclopropyl group.

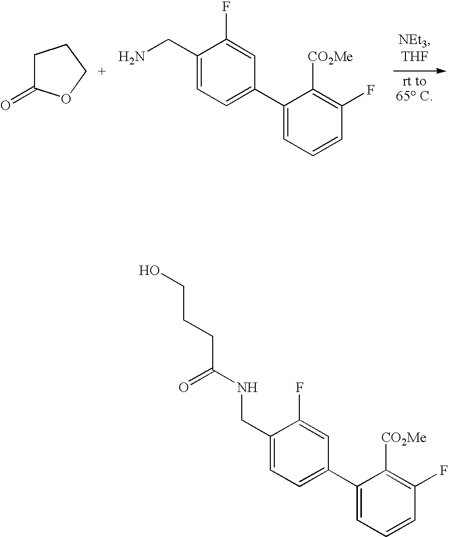

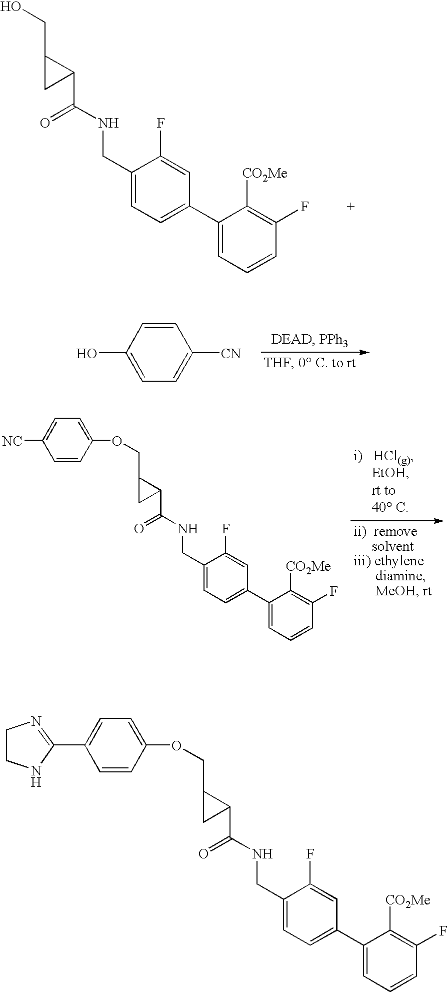

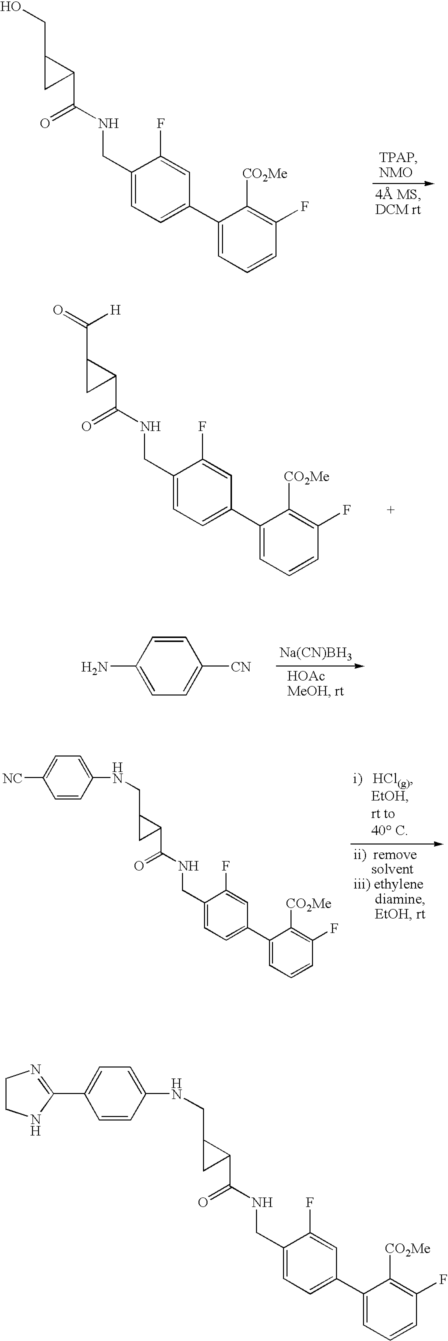

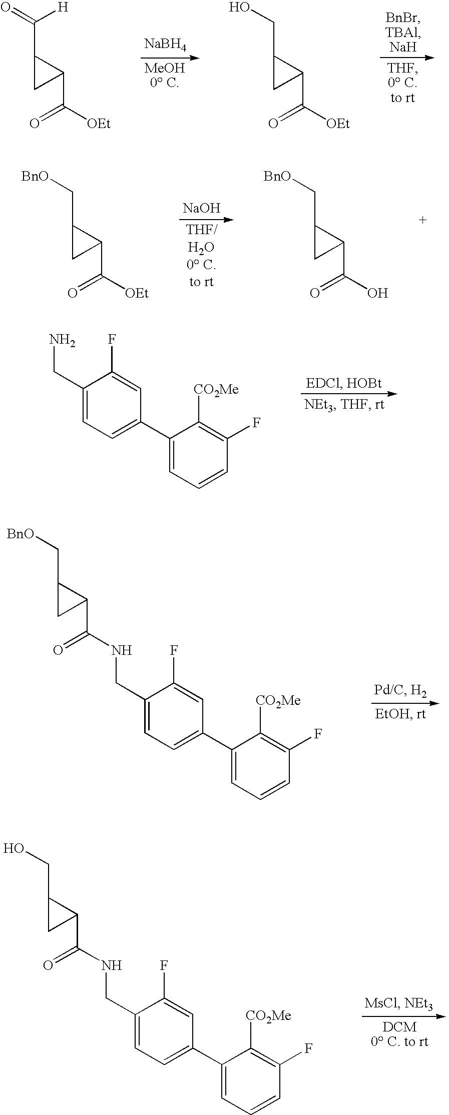

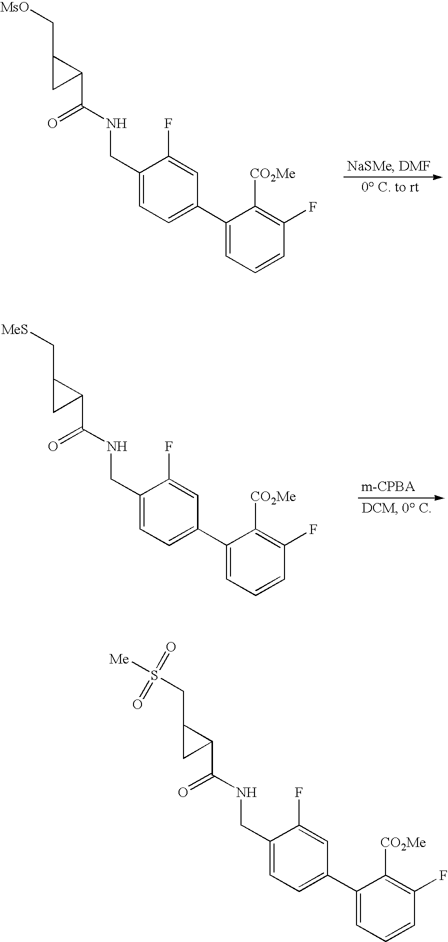

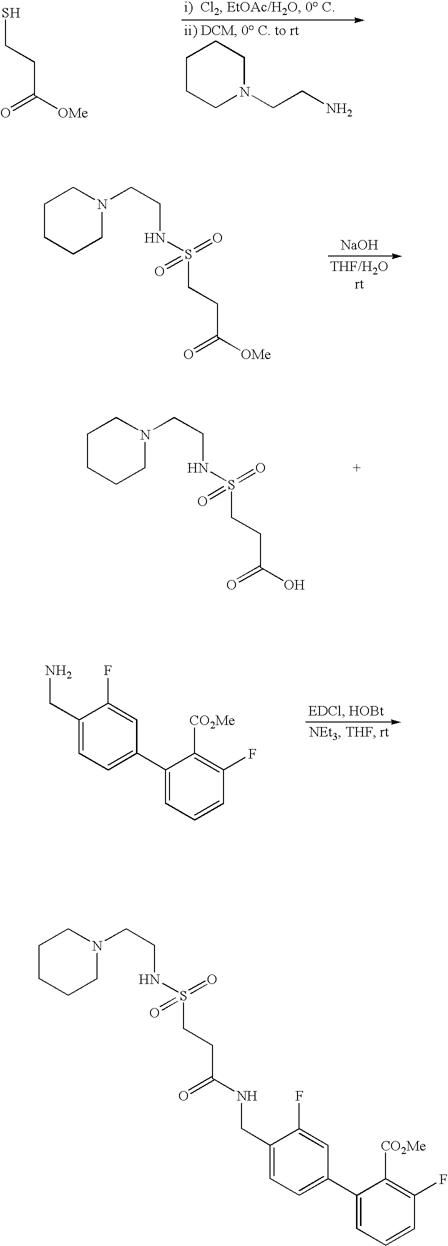

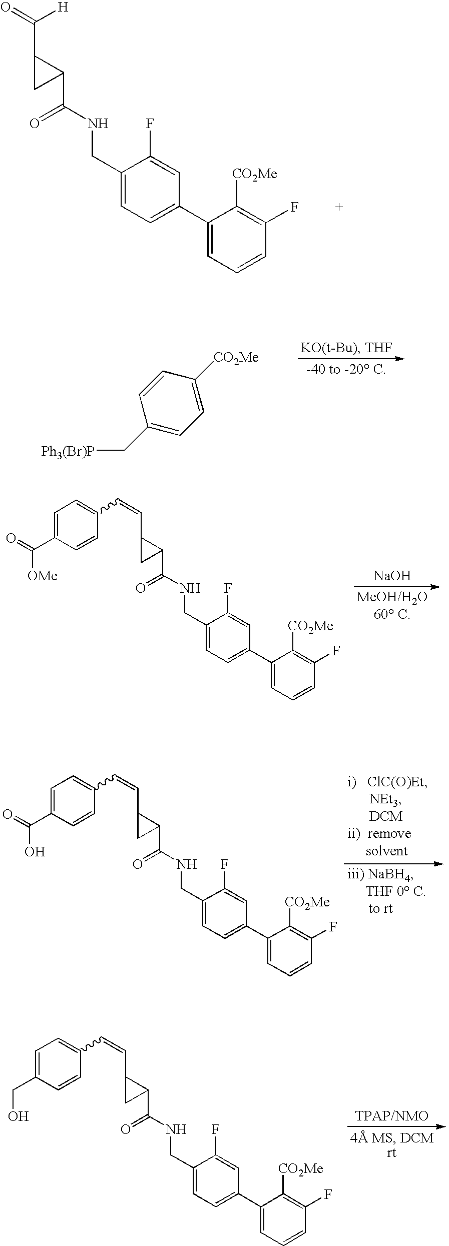

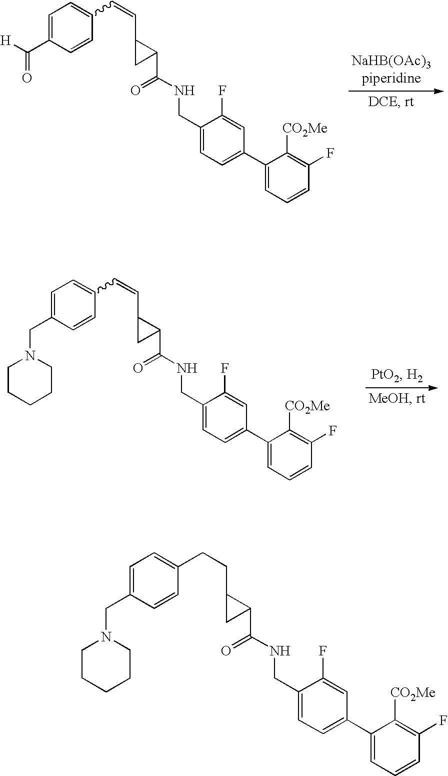

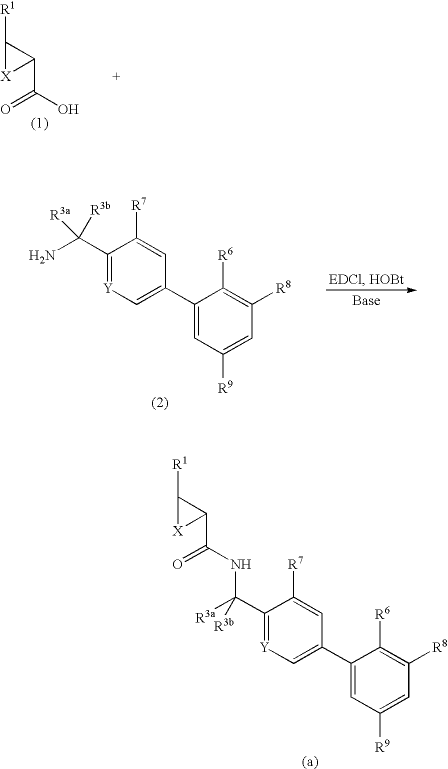

In Scheme 1, the biphenyl derivative (2), assembled according to patent application, WO 03/066577, published 8 Aug. 2003, is reacted with carboxylic acid (1) or a carboxylic acid equivalent using standard peptide coupling reagent combinations, like EDCI/HOBt, in an appropriate solvent, such as THF, with the addition of an appropriate base, like triethylamine (as needed), to provide (a).

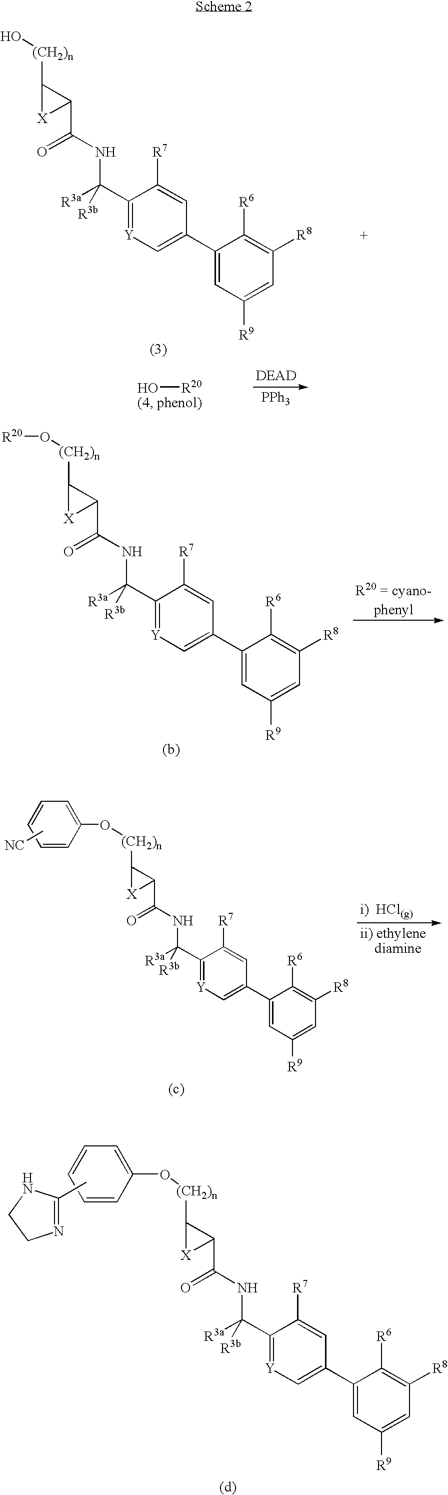

Alternatively, as illustrated in Scheme 2, the alcohol (3), prepared according to Scheme 4, is displaced with a phenol (4), of general formula HOR²⁰, after activation with a standard Mitsunobu reagent combination, such as triphenylphosphine and DEAD, in an appropriate solvent, such as THF, at a temperature between −10 and 50° C. to provide claimed compound (b). In the event that R²⁰ is a cyanophenyl moiety, claimed compound (c (a subset of b)) is further elaborated as shown in Scheme 2.

The cyano group of (Ic) is transformed into an alkyl imidate by the action of a sufficiently strong acid, like HCl, in an appropriate solvent, such as EtOH, and at temperature between 0 and 70° C. This imidate is then reacted with an excess of ethylenediamine, in an appropriate solvent, such as EtOH, at a temperature between 0 and 50° C., to provide (d).

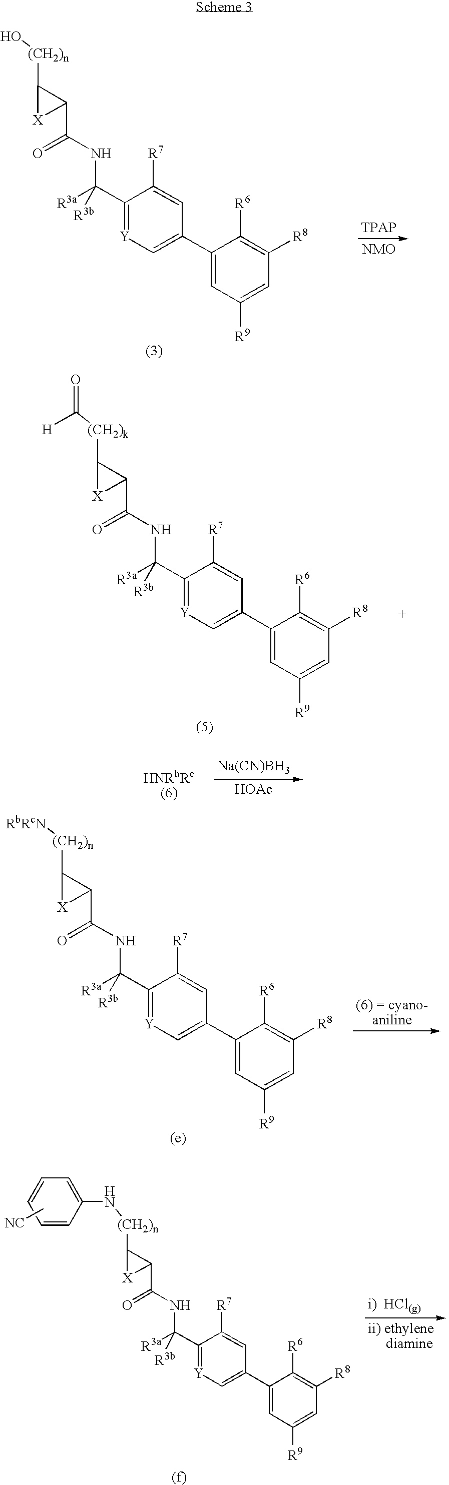

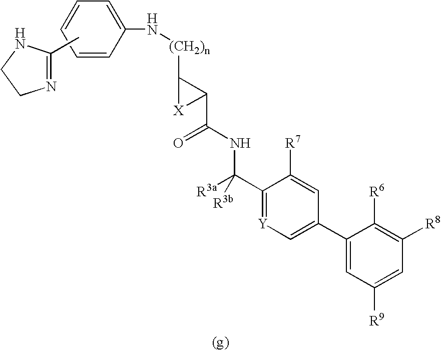

Alternatively, as illustrated in Scheme 3, the alcohol (3), is oxidized to aldehyde (5) by the action of an appropriate oxidant, such as TPAP/NMO, in an appropriate solvent, like DCM, with the addition of a desiccant (as needed), such as 4 Å molecular sieves. Reductive amination between aldehyde (5) and an amine (6), of general formula HNR^{b}R^{c}; or H—R³², employing standard reagent combinations, such as sodium cyanoborohydride with acetic acid, in an appropriate solvent, like MeOH, at a temperature between 0 and 50° C., provides claimed compound (e). In the event that amine (6) is a cyanoaniline, claimed compound (If (a subset of e)) is further elaborated as shown in Scheme 3. The cyano group of (f) is transformed into an alkyl imidate by the action of a sufficiently strong acid, like HCl, in an appropriate solvent, such as EtOH, and at temperature between 0 and 70° C. This imidate is then reacted with an excess of ethylenediamine, in an appropriate solvent, such as EtOH, at a temperature between 0 and 50° C., to provide (g).

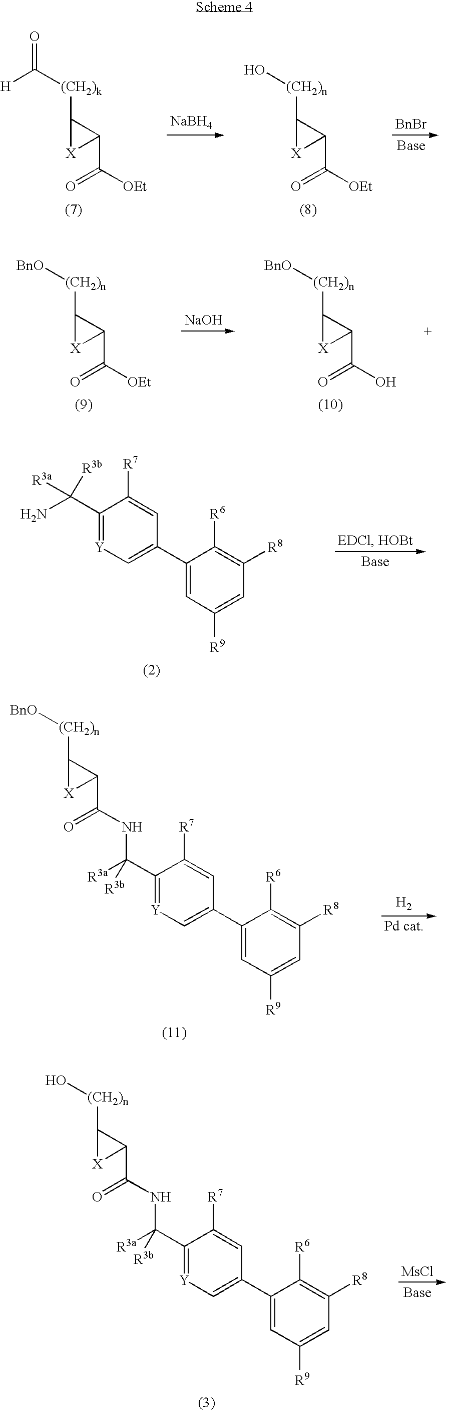

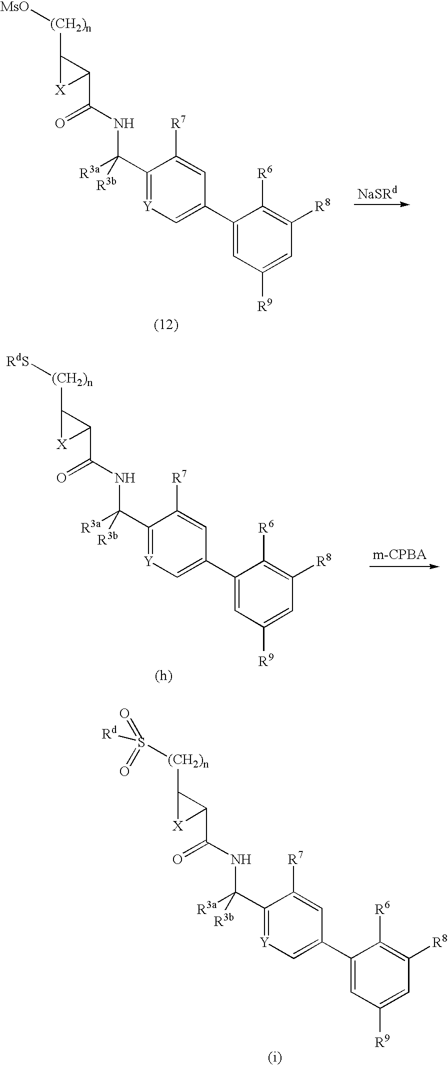

Alternatively, as illustrated in Scheme 4, aldehyde (7) is reduced to alcohol (8) with a reductant, like sodium borohydride, in an appropriate solvent, such as methanol, at a temperature between −20° C. and room temperature. Alcohol (8) is then protected as its benzyl ether by the action of a benzyl halide, in an appropriate solvent, like THF, with the aid of a base, like sodium hydride, and with the addition of a catalyst, such as tetrabutylammonium iodide, at a temperature between 0 and 40° C., to give (9). Alkaline hydrolysis of (9) in a suitable mixture of water and an organic solvent, like THF, at a temperature between 0 and 40° C. yields (10). Carboxylic acid (10) or its carboxylic acid equivalent is reacted with benzylic amine (2), using standard peptide coupling reagent combinations, such as EDCI/HOBt, in an appropriate solvent, such as THF, and in the presence of an appropriate base, such as triethylamine, to provide (11).

The benzylic ether of (11) is reductively cleaved using hydrogen and an appropriate metal catalyst, such as Pd/C, in an appropriate solvent, like ethanol, at a temperature between 0° C. and room temperature, to provide (3). The alcohol of (3) is reacted with a sulfonylation reagent, such as MsCl, in an appropriate aprotic solvent, like DCM, with the addition of a base, such as triethylamine, at a temperature between 0 and 40° C., to provide mesylate (12). This mesylate is then displaced by a metal thiolate, of general formula NaSR^{d}, in an appropriate solvent, like DMF, at a temperature between 0° C. and room temperature, to provide claimed compound (Ih). Thioether (Ih) is then oxidized by an appropriate oxidant, such as M-CPBA, in an appropriate solvent, like DCM, at a temperature between −10 and 30° C., to provide claimed compound (Ii).

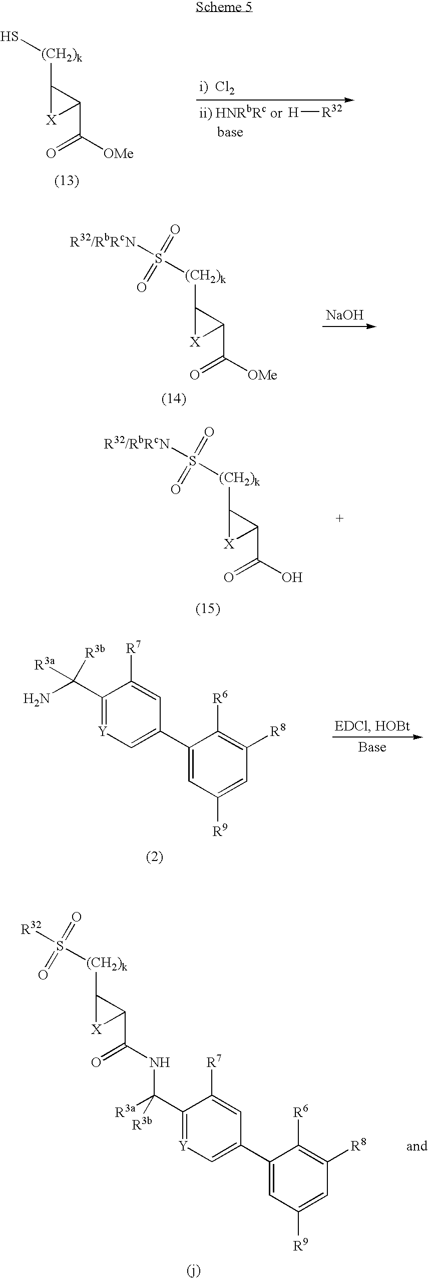

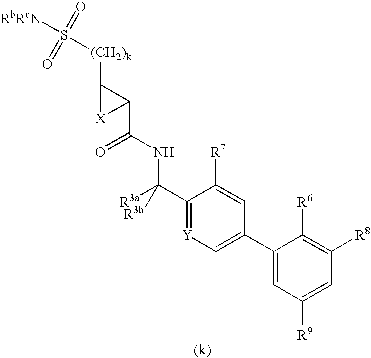

Alternatively, as illustrated in Scheme 5, the thiol of (13) is oxidized to the sulfonyl chloride, using an oxidant, such as chlorine gas, in an appropriate aqueous solvent mixture, such as 1:1 H₂O:EtOAc, at a temperature between −10 and 10° C. This sulfonyl chloride is then reacted with an amine (HNR^{b}R^{c}; or H—R³²), in an appropriate solvent, like DCM, with the addition of a tertiary amine base, such as triethylamine, at a temperature between 0 and 40° C., to provide (14). Alkaline hydrolysis of (14) in a suitable mixture of water and an organic solvent, like THF, at a temperature between 0 and 40° C. yields (15). Carboxylic acid (15) or its carboxylic acid equivalent is reacted with benzylic amine (2), using standard peptide coupling reagent combinations, such as EDCI/HOBt, in an appropriate solvent, such as THF, and in the presence of an appropriate base, such as triethylamine, to provide claimed compounds (j) and (k).

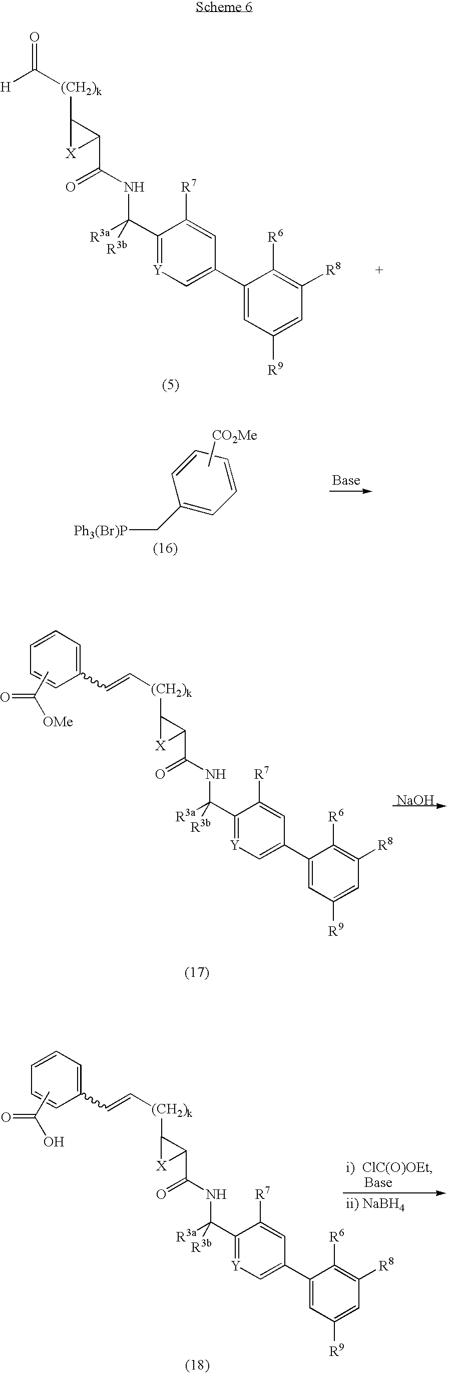

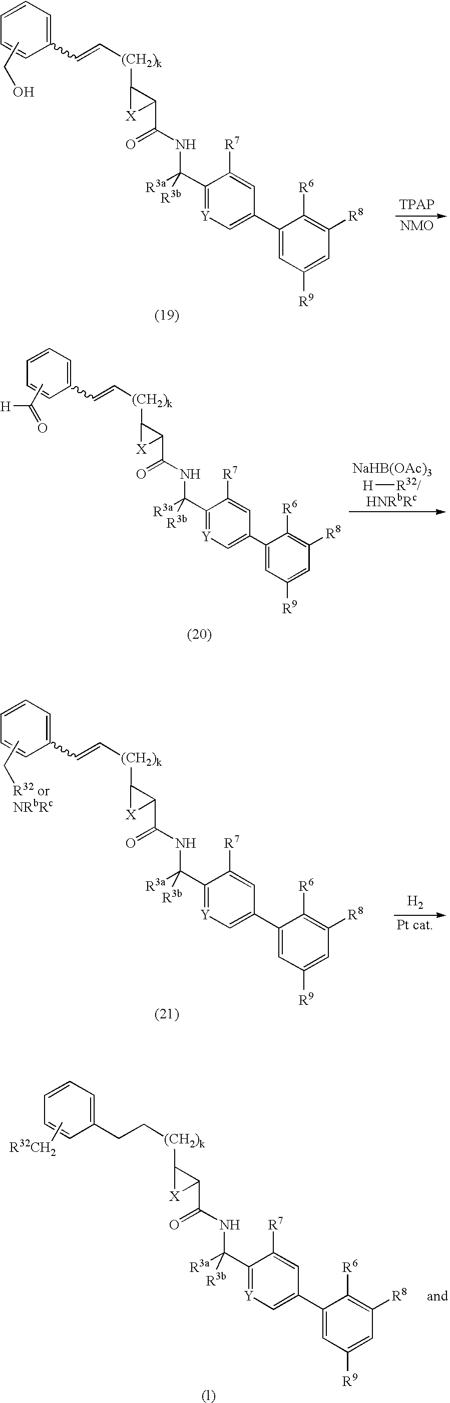

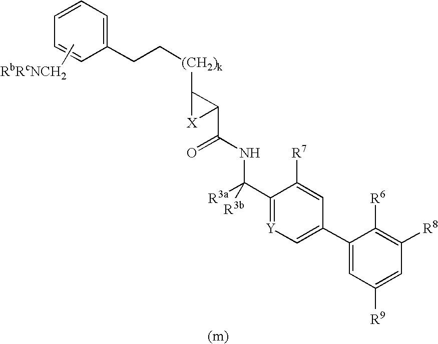

Alternatively, as illustrated in Scheme 6, aldehyde (5) is olefinated by phosphonium reagent (16) in the presence of a strong base, like KO(t-Bu), in an appropriate solvent, such as THF, at a temperature between −40 and −10° C., to provide (17) as a mixture of E and Z olefins. Alkaline hydrolysis of (17) in a suitable mixture of water and an organic solvent, like methanol, at a temperature between 0 and 70° C. yields (18). Carboxylic acid (18) is then converted to its mixed anhydride by reacting with an appropriate chloroformate, like ethyl chloroformate, in the presence of added base, such as triethylamine, in an aprotic sovent, like DCM. The mixed anhydride is then reduced with an appropriate reductant, like sodium borohydride, in an appropriate solvent, like THF, at a temperature between 0 and 40° C., to provide benzylic alcohol (19). Alcohol (19), is oxidized to aldehyde (20) by the action of an appropriate oxidant, such as TPAP/NMO, in an appropriate solvent, like DCM, with the addition of a desiccant (as needed), such as 4A molecular sieves. Reductive amination between aldehyde (20) and an amine (of general formula HNR^{b}R^{c}; or H—R³²), employing standard reagent combinations, such as sodium triacetoxyborohydride, in an appropriate solvent, like DCE, at a temperature between 0 and 50° C., provides compound (21). Reduction of the olefin in (21) with hydrogen, in the presence of an appropriate metal catalyst, like PtO₂, in an appropriate solvent, like methanol, provides claimed compounds (l) and (m).

### EXAMPLE 1

### Methyl 3,3′-difluoro-4′-{[(4-hydroxybutanoyl)amino]methyl}biphenyl-2-carboxylate

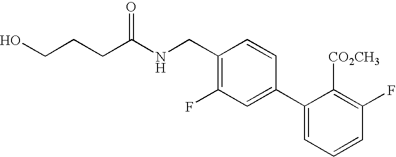

To a solution of methyl 4′-(aminomethyl)-3,3′-difluorobiphenyl-2-carboxylate hydrochloride (0.150 g, 0.478 mmol, prepared according to 21022 PV2) in methanol (1.0 mL) were added triethylamine (0.097 g, 0.96 mmol) and gamma-butyrolactone (0.039 g, 0.45 mmol). The mixture was stirred at room temperature for 3 hours, at 50° C. for 2 hours, and at 60° C. overnight. The mixture was heated at 65° C. for another day. Solvent was removed and the residue was subjected to silica gel chromatography eluted with 1-10% methanol in methylene chloride to provide the title compound. LRMS (ES, M+H⁺): 364. ¹H NMR (CD₃OD, 400 MHz): δ 7.55 (dt, J=8 and 6 Hz, 1H), 7.40 (t, J=8 Hz, 1H), 7.24 (m,2H), 7.11 (m, 2H), 4.45 (s, 2H), 3.69 (s, 3H), 3.57 (t, J=6.4 Hz, 2H), 2.33 (t, J=7.2 Hz, 2H), 1.84 (m, 2H).

### EXAMPLE 2

### Methyl 4′-({[(trans-2-{[4-(4,5-dihydro-1H-imidazol-2-yl)phenoxy]methyl}cyclopropyl)-carbonyl]amino}methyl)-3,3′-difluorobiphenyl-2-carboxylate

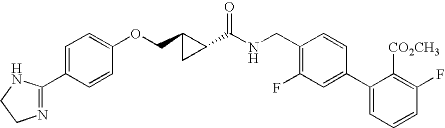

Into a solution of methyl 3,3′-difluoro-4′-[({[trans-2-(hydroxymethyl)cyclopropyl]-carbonyl}amino)methyl]biphenyl-2-carboxylate (0.150 g, 0.40 mmol, prepared according to the procedure in example 4) and 4-cyanophenol (0.052 g, 0.44 mmol) in THF (2.0 mL) was added triphenylphosphine (0.157 g, 0.60 mmol), and the resulting mixture was cooled to 0° C. DEAD (0.105 g, 0.60 mmol) was added to the mixture. Additional DEAD, triphenyl phosphine, and 4-cyanophenol were added periodically during 24 hours to drive the reaction to completion. The mixture was concentrated and the residue was subjected to silica gel chromatography eluted with 0-50% ethyl acetate in hexane to provide methyl 4′-{[({trans-2-[(4-cyanophenoxy)methyl]cyclopropyl}carbonyl)amino]methyl}-3,3′-difluorobiphenyl-2-carboxylate.

A solution of the above material (0.100 mg, 0.21 mmol) in ethanol (2 mL) was saturated with anhydrous HCl, and the resulting solution was stirred at 40° C. for 1 hour and then cooled to room temperature for overnight stirring. Solvent was removed, and the residue was dissolved in methanol (2 mL). Ethylene diamine (0.025 g, 0.42 mmol) was added and the resulting solution was stirred at room temperature overnight. Upon completion of the reaction, the mixture was concentrated and the residue was purified by reverse phase chromatography to yield the title compound. HRMS (ES, M+H³⁰): calc'd 519.1970, measured 520.2038. ¹H NMR (CD₃OD, 300 MHz): δ 7.81 (d, J=9 Hz, 2H), 7.56 (dt, J=8.1 and 5.7 Hz, 1H), 7.35 (t, J=8.4 Hz, 1H), 7.24 (m, 2H), 7.13 (m, 4H), 4.47 (s, 2H), 4.21 (dd, J=10.5 and 5.7 Hz, 1H), 4.04 (s, 4H), 3.94 (dd, J=10.5 and 7.2 Hz, 1H), 3.68 (s, 3H), 1.80 (m, 2H), 1.23 (m, 1H), 0.97 (m,1H).

### EXAMPLE 3

### Methyl 4′-[({[trans-2-({[4-(4,5-dihydro-1H-imidazol-2-yl)phenyl]amino}methyl)-cyclopropyl]carbonyl}amino)methyl]-3,3′-difluorobiphenyl-2-carboxylate

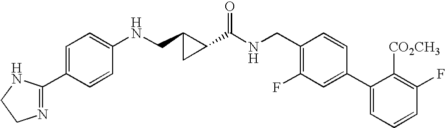

Methyl 3,3′-difluoro-4′-[({[trans-2-(hydroxymethyl)cyclopropyl]carbonyl}amino)-methyl]biphenyl-2-carboxylate (0.47 g, 1.25 mmol) and crushed 4A molecular sieves were stirred in dry methylene chloride (10 mL) under nitrogen, and 4-methylmorpholine N-oxide (0.191 g, 1.63 mmol) was added. After 15 minutes, TPAP (0.044 g, 0.13 g) was added and the resulting mixture was stirred for 30 minutes. The mixture was filtered through a pad of celite and the celite cake was washed with chloroform. The filtrate was washed twice with water and once with brine. The organic layer was concentrated, and the residue was subjected to silica gel chromatography eluted with 0-50% ethyl acetate in hexane to afford methyl 3,3′-difluoro-4′-[({[trans-2-formylcyclopropyl]carbonyl}amino)methyl]-biphenyl-2-carboxylate as a pale yellow oil.

A solution of the above material (0.100 g, 0.27 mmol), 4-aminobenzonitrile (0.029 g, 0.24 mmol) and acetic acid (0.088 g, 1.47 mmol) in methanol (1.0 mL) was stirred for 30 minutes. Sodium cyanoborohydride (0.039 g, 0.61 mmol) was added, and the mixture was stirred for 30 minutes. The reaction was quenched with by addition of aqueous sodium bicarbonate, and the product was extracted with ethyl acetate (×3). The combined organic extracts were dried over sodium sulfate, filtered, and concentrated, and the residue was subjected to silica gel chromatography eluted with 0-50% ethyl acetate in hexane to yield methyl 4′-({[(trans-2-{[(4-cyanophenyl)amino]methyl}cyclopropyl)-carbonyl]amino}methyl)-3,3′-difluorobiphenyl-2-carboxylate.

The above material (0.042 g, 0.088 mmol) was dissolved in dry ethanol (2.0 mL), which was subsequently saturated with HCl, and then the solution was heated at 50° C. Upon the formation of the desired imidate, solvent was removed and the residue was again dissolved in ethanol (2.0 mL). Ethylene diamine (0.011 g, 0.18 mmol) was added, and the solution was heated at 50° C. for 1 hour. Solvent was removed and the residue was purified by reverse phase chromatography to provide the title compound as a TFA salt. LRMS (ES, M+H³⁰): 519. ¹H NMR (CD₃OD, 400 MHz): δ 7.61 (d, J=8.8 Hz, 2H), 7.56 (m, 1H), 7.33 (t, J=8 Hz, 1H), 7.25 (m, 2H), 7.09 (m, 2H), 6.74 (d, J=9.2 Hz, 2H), 4.45 (s, 2H), 3.96 (s, 4H), 3.68 (s, 3H), 3.19 (m, 2H), 1.64 (m, 2H), 1.15 (m, 1H), 0.86 (m, 1H).

### EXAMPLE 4

### Methyl 3,3′-difluoro-4′-{[({trans-2-[(methylsulfonyl)methyl]cyclopropyl}carbonyl)-amino]methyl}biphenyl-2-carboxylate

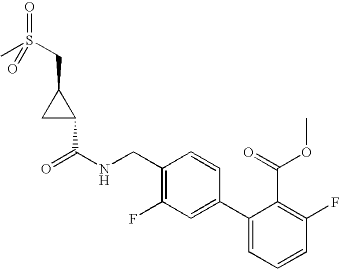

To a solution of ethyl trans-2-formyl-1-cyclopropanecarboxylate (2.000 g, 14.07 mmol) in methanol (70 mL) at 0° C. was added sodium borohydride (0.532 g, 14.07 mmol). After stirring for 1 hour, solvent was removed and the residue was partitioned between ether (100 mL) and a 1:1 mixture (50 mL) of brine and 1N HCl. The organic layer was washed three times with half brine and once with brine. The organic solution was dried over sodium sulfate, filtered, and concentrated to give ethyl trans-2-(hydroxymethyl)cyclopropanecarboxylate.

To a solution of the above material (1.04 g, 7.21 mmol) in THF (50 mL) were added TBAI (0.133 g, 0.36 mmol) and benzyl bromide (1.85 g, 10.8 mmol). The mixture was cooled to 0° C. and sodium hydride (0.225 g, 9.38 mmol) was added. After stirring at 0° C. for 1 hour, the mixture was warmed to room temperature for overnight stirring. The mixture was diluted with ethyl acetate and washed three times with half brine and once with brine. The organic layer was dried over sodium sulfate, filtered, and concentrated. The residue was subjected to silica gel chromatography eluted with 0-10% methanol in methylene chloride to yield ethyl trans-2-[(benzyloxy)methyl]cyclopropanecarboxylate.

To a solution of the above material (1.141 g, 4.87 mmol) in THF (26 mL) at 0° C. was added 1N NaOH (5.6 mL). After 1 hour at 0° C., the solution was stirred at room temperature for 4 days. THF was removed under reduced pressure and the residual aqueous solution was washed with ether 4 times. 1N HCl (5.70 mL) was added to the aqueous solution, and the product was extracted with two volumes of methylene chloride. The methylene chloride solutions were dried over sodium sulfate, filtered, and concentrated to give trans-2-[(benzyloxy)methyl]cyclopropanecarboxylic acid.

Into a solution of the above acid (0.542 g, 2.63 mmol), methyl 4′-(aminomethyl)-3,3′-difluorobiphenyl-2-carboxylate hydrochloride (0.825 g, 2.63 mmol) and 1-hydroxybenzotriazole hydrate (0.081 g, 0.53 mmol) in THF (28.2 mL) were added triethylamine (0.585 g, 5.78 mmol) and 1-(3-dimethylaminopropyl)-3-ethylcarbodiimide hydrochloride (0.605 g, 3.15 mmol). After stirring overnight, the mixture was diluted with ethyl acetate and washed with 1N HCl, 5% sodium bicarbonate, and brine. The organic layer was dried over sodium sulfate, filtered, and concentrated. The residue was subjected to silica gel chromatography eluted with 20-70% ethyl acetate in hexane to yield methyl 4′-{[({trans-2-[(benzyloxy)methyl]cyclopropyl}carbonyl)amino]methyl}-3,3′-difluorobiphenyl-2-carboxylate.

A solution of the above material (0.536 g, 1.15 mmol) in ethanol (10.8 mL) was purged with nitrogen prior to the addition of 10% Pd/C catalyst (0.037 g). The mixture was again purged with nitrogen and then with hydrogen from a balloon. For 5 days, additional 10% Pd/C was added periodically to drive the reaction to completion. The catalyst was filtered off through a pad of celite and washed with ethanol. The filtrate was concentrated and the residue was subjected to silica gel chromatography eluted with 45-100% ethyl acetate in hexane to provide methyl 3,3′-difluoro-4′-[({[trans-2-(hydroxymethyl)cyclopropyl]carbonyl}amino)methyl]biphenyl-2-carboxylate.

Into a solution of the above material (0.110 g, 0.293 mmol) in methylene chloride (1.5 mL) at 0° C. was added triethylamine (0.059 g, 0.59 mmol) followed by methanesulfonyl chloride (0.067 g, 0.59 mmol). The solution was warmed to room temperature and stirred for 1.5 hours. Solvent was removed. The residue was dissolved in ethyl acetate and washed twice with water (10 mL) and once with brine (10 mL). The organic layer was dried over sodium sulfate, filtered and concentrated to yield methyl 3,3′-difluoro-4′-({[(trans-2-{[(methylsulfonyl)oxy]methyl}cyclopropyl)carbonyl]amino}methyl)-biphenyl-2-carboxylate.

A solution of the above material (0.12 g, 0.27 mmol) in DMF (1.4 mL) under nitrogen was cooled to 0° C. Sodium thiomethoxide (0.041 g, 0.59 mmol) was added, and the resulting mixture was stirred at 0° C. for 10 minutes and at room temperature for 1.5 hours. The mixture was diluted with ethyl acetate (80 mL) and washed with water (2×15 mL) and brine (15 mL). The organic layer was dried over sodium sulfate, filtered and concentrated to give methyl 3,3′-difluoro-4′-{[({trans-2-[(methylthio)-methyl]cyclopropyl}carbonyl)amino]methyl}biphenyl-2-carboxylate.

Into a solution of the above material (0.049 g, 0.12 mmol) in methylene chloride (0.7 mL) at 0° C. was added mCPBA (0.044 g, 0.26 mmol). After 40 minutes at 0° C., ammonia gas was bubbled through the solution. Solvent was removed, and the residue was dissolved in methylene chloride and washed three times with aq. 10% sodium carbonate (15 mL). The organic layer was concentrated and the residue was subjected to silica gel chromatography eluted with 1-5% methanol in methylene chloride to provide the title compound. LRMS (ES, M+H⁺): 438 ¹H NMR (CD₃OD, 400 MHz): δ 7.55 (dt, J=8 and 5.6 Hz, 1H), 7.41 (t, J=8 Hz, 1H), 7.24 (m, 2H), 7.12 (m, 2H), 4.48 (m, 2H), 3.69 (s, 3H), 3.23 (m, 1H), 3.10 (m, 1H), 2.97 (s, 3H), 1.78 (m, 1H), 1.66 (m, 1H), 1.28 (m, 1H), 0.96 (m, 1H).

### EXAMPLE 5

### Methyl 3,3′-difluoro-4′-{[(3-{[(2-piperidin-1-ylethyl)amino]sulfonyl}propanoyl)am ino]-methyl}biphenyl-2-carboxylate

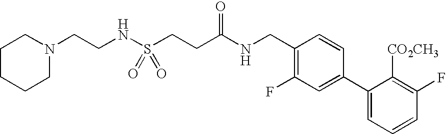

A mixture of ethyl acetate (10 mL) and water (10 mL) was cooled to 0° C., and methyl 3-mercaptopropionate (0.155 g, 1.29 mmol) was added. Chlorine gas was bubbled through the solution for 2 minutes until the solution became bright yellow. The solution was partitioned between methylene chloride (100 mL) and water (100 mL), and the organic layer was dried over sodium sulfate, filtered, and concentrated to give a clear oil. The oil was dissolved in methylene chloride (10 mL) and the solution was cooled to 0° C. N-(2-aminoethyl)piperidine (0.496 g, 3.87 mmol) was added slowly over 2 minutes, and the resulting mixture was slowly warmed to room temperature for overnight stirring. Solvent was removed and the residue was diluted with ethyl acetate. The solution was washed three times with saturated sodium bicarbonate and then dried over sodium sulfate, filtered, and concentrated. The residue was subjected to silica gel chromatography eluted with 1-15% methanol (with 10% NH₄OH) in methylene chloride to get methyl 3-{[(2-piperidin-1-ylethyl)amino]sulfonyl}propanoate as a clear oil. Into a solution of the above material (0.177 g, 0.64 mmol) in THF (6.4 mL) was added 1N NaOH (1.27 mL). After overnight stirring, the THF was removed under reduced pressure. The aqueous residue was diluted with water and washed three times with diethyl ether. 1N HCl (1.91 mL) was added to the aqueous solution, and the resulting solution was lyophilized to yield 3-{[(2-piperidin-1-ylethyl)amino]-sulfonyl}propanoic acid.

Into a suspension of the above material (0.124 g, 0.30 mmol), methyl 4′-(aminomethyl)-3,3′-difluorobiphenyl-2-carboxylate (0.082 g, 0.30 mmol), 1-hydroxybenzotriazole hydrate (0.046 g, 0.30 mmol) and triethylamine (0.066 g, 0.65 mmol) in DMF (3.0 mL) was added 1-(3-dimethylaminopropyl)-3-ethylcarbodiimide hydrochloride (0.068 g, 0.36 mmol). The resulting mixture was stirred at room temperature for 2 hours and then heated at 40° C. for 4 hours. The mixture was cooled to ambient temperature and solvent was removed. The residue was diluted with ethyl acetate and washed three times with saturated sodium bicarbonate and once with brine. The organic layer was dried over sodium sulfate, filtered, and concentrated. The residue was subjected to silica gel chromatography eluted with 1-15% methanol (with 10% NH₄OH) in methylene chloride to provide the title compound. LRMS (ES, M+H³⁰): 524.1H NMR(CD₃OD, 400 MHz): δ 7.55 (dt, J=8 and 6 Hz, 1H), 7.43 (t,J=8 Hz, 1H), 7.24 (m, 2H), 7.12 (m, 2H), 4.70 (s, 2H), 3.69 (s, 3H), 3.40 (t, J=7.2 Hz, 2H), 3.19 (t, J=6.8 Hz, 2H), 2.73 (t, J=7.2 Hz, 2H), 2.48 (t, J=6.8 Hz, 2H), 2.45 (m, 4H), 1.59 (m, 4H), 1.46 (m, 4H).

### EXAMPLE 6

### Methyl 3,3′-difluoro-4′-({[(trans-2-{2-[4-(piperidin-1-ylmethyl)phenyl]ethyl}-cyclopropyl)carbonyl]amino}methyl)biphenyl-2-carboxylate

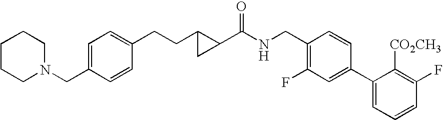

Into a suspension of [4-(methoxycarbonyl)benzyl](triphenyl)phosphonium bromide (1.316 g, 2.68 mmol) in THF (10 mL) at −40° C. was added potassium t-butoxide (0.451 g, 4.02 mmol). After 30 minutes, a solution of methyl 3,3′-difluoro-4′-[({[trans-2-formylcyclopropyl]carbonyl}amino)-methyl]biphenyl-2-carboxylate (0.500 g, 1.34 mmol, prepared according to example 3) in THF (6 mL) was added dropwise, and the resulting solution was warmed to −20° C. After 30 minutes, solvent was removed, and the residue was subjected to silica gel chromatography to provide a mixture of E/Z isomers of methyl 3,3′-difluoro-4′-({[(trans-2-{2-[4-(methoxycarbonyl)phenyl]vinyl}cyclopropyl)carbonyl]-amino}methyl)biphenyl-2-carboxylate. The isolated material was dissolved in a 1:1 mixture (10 mL) of methanol and water, and 1N NaOH (1.34 mL) was added. The resulting solution was heated at 60° C. for 2 hours. Methanol was removed under reduced pressure, and the remaining aqueous solution was acidified by addition of 1N HCl. The product was extracted with ethyl acetate and the combined extracts were concentrated to provide 4-(2-{trans-2-[({[3,3′-difluoro-2′-(methoxycarbonyl)biphenyl-4-yl]methyl}-amino)carbonyl]cyclopropyl}vinyl)benzoic acid.

Into a solution of the above material (0.520 g, 1.06 mmol) and triethylamine (0.214 g, 2.12 mmol) in methylene chloride (20 mL) was added ethylchloroformate (0.145 g, 1.06 mmol) dropwise. After 1 hour of stirring, the mixture was concentrated and the residue was dissolved in THF (20 mL). The solution was cooled to 0° C., and sodium borohydride (0.080 g, 2.12 mmol) was added. The resulting mixture was warmed to room temperature and stirred for 5 hours. The reaction was quenched with aq. ammonium chloride and extracted with ethyl acetate. The organic layer was washed with brine, dried over sodium sulfate, filtered, and concentrated. The residue was subjected to silica gel chromatography eluted with 20-70% ethyl acetate in hexane to provide methyl 3,3′-difluoro-4′-({[(trans-2-{2-[4-(hydroxymethyl)phenyl]vinyl}cyclopropyl)carbonyl]amino}methyl)biphenyl-2-carboxylate as a white foam.

Into a solution of the above material (0.22 g, 0.46 mmol) and 4-methylmorpholine N-oxide (0.070 g, 0.60 mmol) in methylene chloride (5.0 mL) were added dry crushed 4A molecular sieves (0.5 g). After 5 minutes, TPAP was added in small portions and the resulting mixture was stirred for 5 hours. The mixture was filtered through a pad of celite and the celite cake was washed with ethyl acetate. The filtrate was concentrated and the residue was subjected to silica gel chromatography eluted with 20-60% ethyl acetate in hexane to afford methyl 3,3′-difluoro-4′-{[({trans-2-[2-(4-formylphenyl)-vinyl]cyclopropyl}carbonyl)amino]methyl}biphenyl-2-carboxylate.

The above material (0.089 g, 0.187 mmol) and sodium triacetoxyborohydride (0.127 g, 0.60 mmol) were dissolved in dichloroethane (2 mL), and piperidine (0.079 g, 0.92 mmol) was added. After overnight stirring, the mixture was diluted with methylene chloride and washed with water and brine. The organic layer was dried over sodium sulfate, filtered and concentrated. The residue was subjected to silica gel chromatography eluted with 10% methanol in methylene chloride to provide methyl 3,3′-difluoro-4′-({[(trans-2-{2-[4-(piperidin-1-ylmethyl)phenyl]vinyl}cyclopropyl)carbonyl]-amino}methyl)biphenyl-2-carboxylate.

Into a solution of the above material (0.035 g, 0.064 mmol) in methanol (1.0 mL) was added platinum(IV) oxide (3.6 mg, 0.02 mmol). The solution was purged with hydrogen from a balloon. After 20 minutes, the mixture was filtered through a cotton layer, and the filtrated was concentrated. The residue was purified by reverse phase chromatography eluted with 15-80% acetonitrile in water to provide the title compound as a TFA salt. LRMS (ES, M+H³⁰): 547. ¹H NMR (CD₃OD, 400 MHz): 7.56 (m, 1H), 7.41-7.22 (m, 7H), 7.14 (m, 2H), 4.45 (m, 2H), 4.22 (s, 2H), 3.68 (s, 3H), 3.41 (bd, J=12.4 Hz, 2H), 2.91 (bt, J=12.4 Hz, 2H), 2.77 (m, 2H), 1.93-1.40 (m, 9H), 1.22 (m, 1H), 1.06 (m, 1H), 0.66 (m, 1H).

TABLE A through TABLE G below, provide compounds of Formula (Ia), (Ib) or (Ic) that have been prepared by the method described above. The binding affinity of these compounds for BK receptor B1 ranges from 0.4 nM to 10 μM.

TABLE A

| | |
|---|---|
| | A |
| 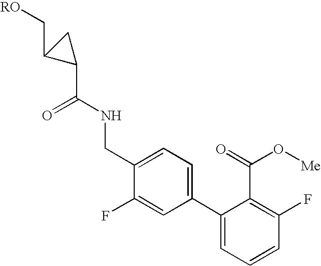 | |

| | | LRMS |
|---|---|---|
| Example | R | (M + H⁺) |
| 7 | Benzyl | 466 |
| 8 | H | 376 |
| 9 | 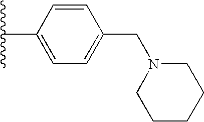 | 549 |
| 10 | 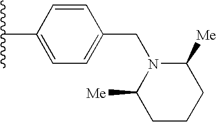 | 577 |
| 11 | 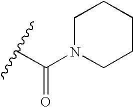 | 487 |
| 12 | 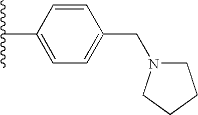 | 535 |
| 13 | 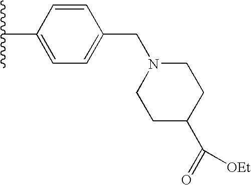 | 621 |
| 14 | 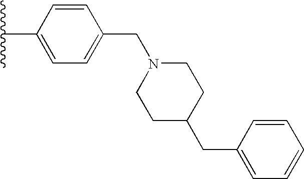 | 639 |
| 15 | 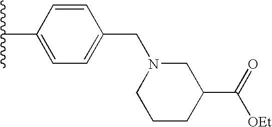 | 621 |
| 16 | 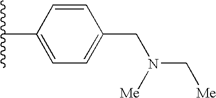 | 523 |
| 17 | 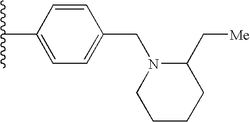 | 577 |
| 18 | 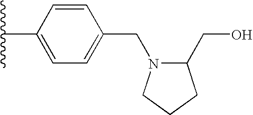 | 565 |
| 19 | 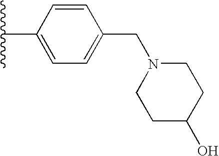 | 565 |
| 20 | 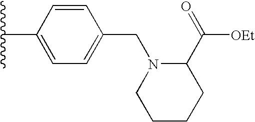 | 621 |
| 21 | 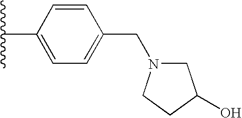 | 551 |
| 22 | 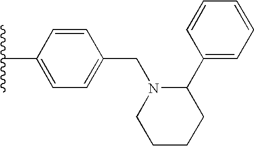 | 625 |
| 23 | 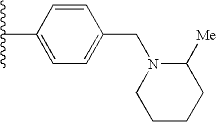 | 563 |
| 24 | 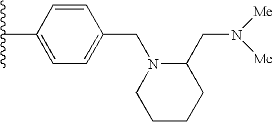 | 606 |
| 25 | 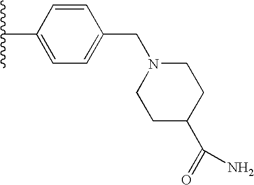 | 592 |
| 26 | 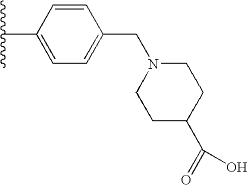 | 593 |
| 27 | 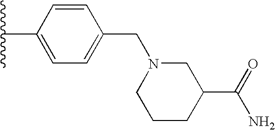 | 662 |
| 28 | 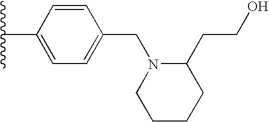 | 593 |
| 29 | 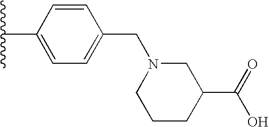 | 593 |
| 30 | 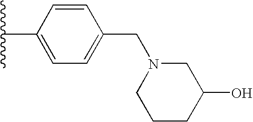 | 565 |
| 31 | 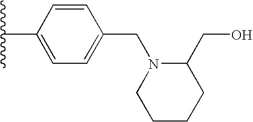 | 579 |
| 32 | 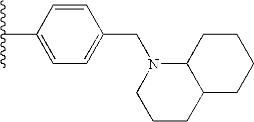 | 603 |
| 33 | 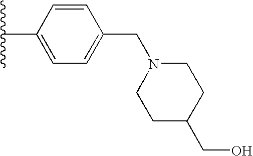 | 579 |
| 34 | 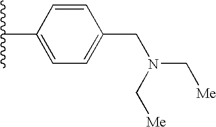 | 537 |
| 35 | 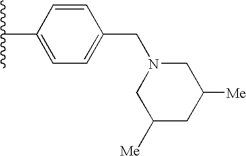 | 577 |
| 36 | 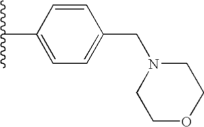 | 551 |
| 37 | 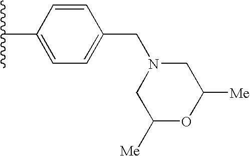 | 579 |
| 38 | 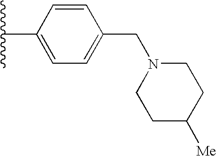 | 563 |
| 39 | 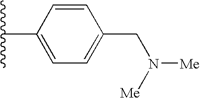 | 509 |
| 40 | 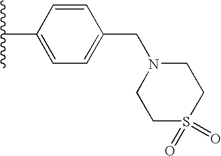 | 599 |
| 41 | 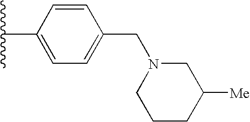 | 563 |
| 42 | 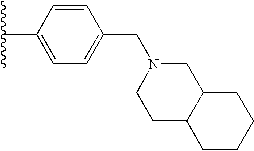 | 603 |
| 43 | 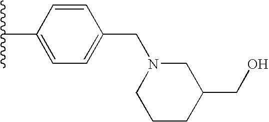 | 579 |
| 44 | 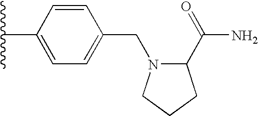 | 578 |
| 45 | 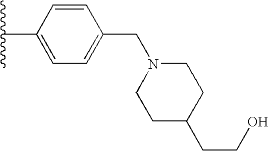 | 593 |
| 46 | 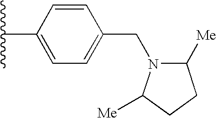 | 563 |
| 47 | 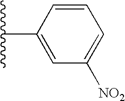 | 497 |
| 48 | 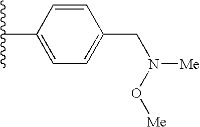 | 525 |

TABLE B

| | |
|---|---|
| | B |
| 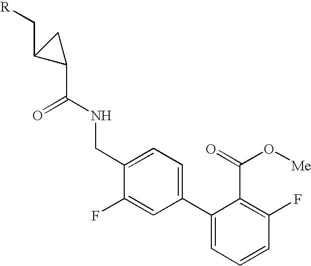 | |

| | | LRMS |
|---|---|---|
| Example | R | (M + H⁺) |
| 49 | 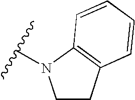 | 477 |
| 50 | 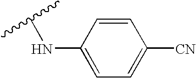 | 476 |
| 51 | 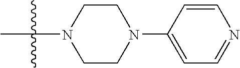 | 521 |
| 52 | 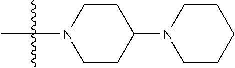 | 526 |
| 53 | 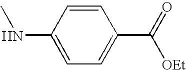 | 523 |
| 54 | 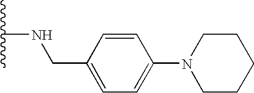 | 548 |
| 55 | 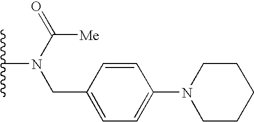 | 590 |
| 56 | 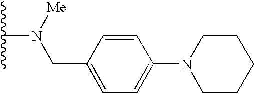 | 562 |
| 57 | 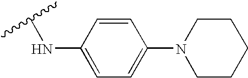 | 534 |
| 58 | 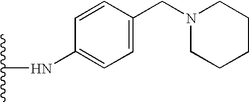 | 548 |
| 59 | 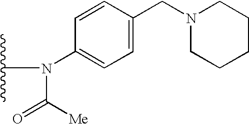 | 590 |
| 60 | 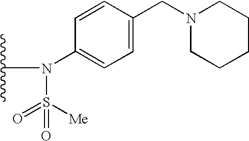 | 626 |
| 61 | 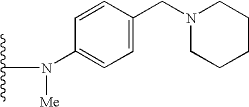 | 562 |
| 62 | 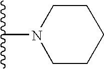 | 443 |
| 63 | 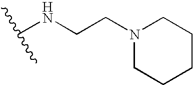 | 486 |
| 64 | 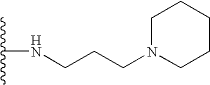 | 500 |
| 65 | 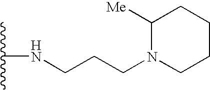 | 514 |
| 66 | 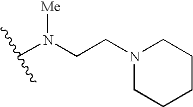 | 500 |
| 67 | 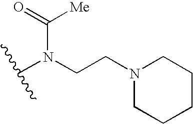 | 528 |
| 68 | 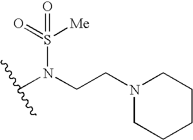 | 564 |
| 69 | 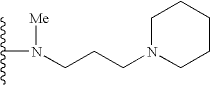 | 514 |
| 70 | 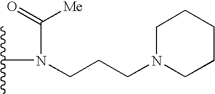 | 542 |
| 71 | 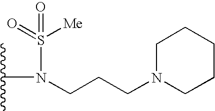 | 578 |
| 72 | 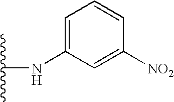 | 496 |
| 73 | 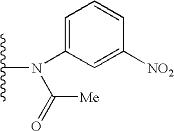 | 538 |
| 74 | 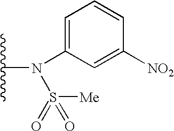 | 574 |
| 75 | 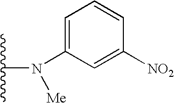 | 510 |
| 76 | 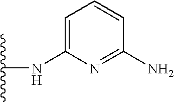 | 467 |
| 77 | 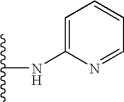 | 452 |
| 78 | 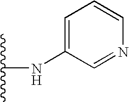 | 452 |
| 79 | 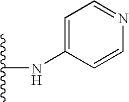 | 452 |
| 80 | 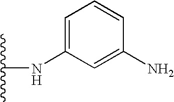 | 466 |
| 81 | 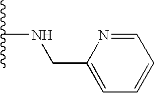 | 466 |
| 82 | 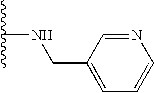 | 514 |
| 83 | 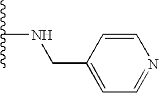 | 466 |
| 84 | 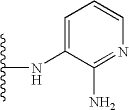 | 467 |
| 85 | 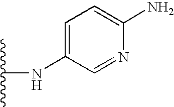 | 467 |
| 86 | 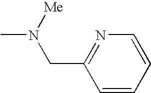 | 514 |
| 87 | 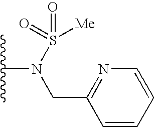 | 544 |
| 88 | 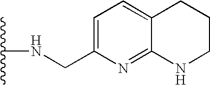 | 521 |

TABLE C

| | |
|---|---|
| | C |
| 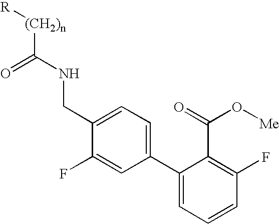 | |

| | | | LRMS |
|---|---|---|---|
| Example | R | n | (M + H⁺) |
| 89 | 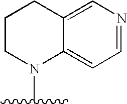 | 3 | 480 |
| 90 | 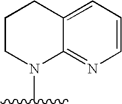 | 3 | 480 |
| 91 | 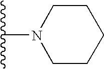 | 3 | 431 |
| 92 | 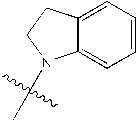 | 2 | 451 |
| 93 | 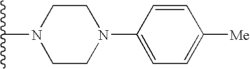 | 5 | 550 |
| 94 | 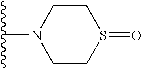 | 3 | 465 |
| 95 | 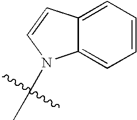 | 3 | 463 |
| 96 | 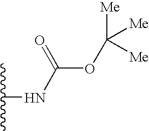 | 4 | 477 |
| 97 | 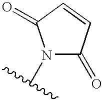 | 3 | 443 |
| 98 | 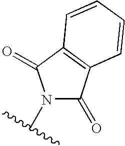 | 3 | 493 |
| 99 | 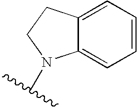 | 3 | 465 |
| 100 | 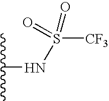 | 3 | 495 |
| 101 | 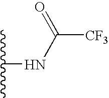 | 3 | 459 |
| 102 | 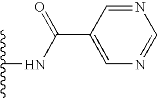 | 3 | 469 |
| 103 | 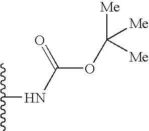 | 3 | 463 |
| 104 | SO₃H | 3 | 428 |
| 105 | SO₃H | 2 | 414 |
| 106 | 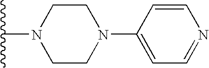 | 3 | 573 |
| 107 | 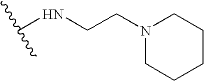 | 3 | 538 |
| 108 | SO₂NH₂ | 3 | 427 |

TABLE D

| | |
|---|---|
| | D |
| 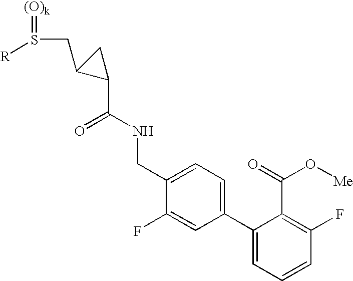 | |

| | | | LRMS |
|---|---|---|---|
| Example | R | k | (M + H⁺) |
| 109 | Me | 0 | 406 |
| 110 | Et | 0 | 420 |
| 111 | 2-propyl | 0 | 433 |
| 112 | Phenyl | 0 | 468 |
| 113 | 2-pyridinyl | 0 | 469 |
| 114 | 4-pyridinyl | 0 | 469 |
| 115 | Et | 2 | 452 |
| 116 | 2-propyl | 2 | 1001 |
| 117 | Phenyl | 2 | 1001 |
| 118 | 2-pyridinyl | 2 | 500 |
| 119 | 4-pyridinyl | 2 | 501 |
| 120 | 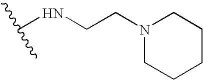 | 2 | 550 |
| 121 | 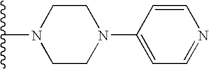 | 2 | 585 |

TABLE E

| | |
|---|---|
| | E |
| 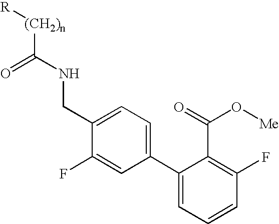 | |

| | | | LRMS |
|---|---|---|---|
| Example | R | n | (M + H⁺) |
| 122 | 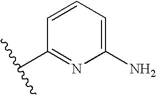 | 3 | 440 |
| 123 | 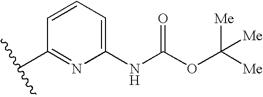 | 3 | 540 |
| 124 | 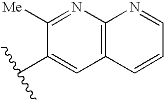 | 2 | 476 |
| 125 | 2-pyridinyl | 2 | 411 |
| 126 | 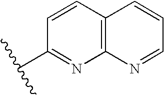 | 3 | 476 |
| 127 | 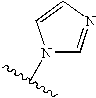 | 7 | 470 |
| 128 | 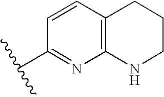 | 2 | 466 |
| 129 | 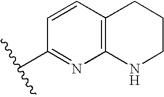 | 4 | 494 |
| 130 | 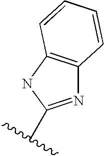 | 4 | 478 |
| 131 | 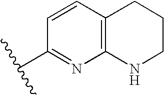 | 3 | 480 |
| 132 | 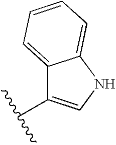 | 3 | 463 |
| 133 | 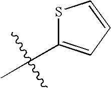 | 3 | 430 |
| 134 | CO₂Me | 3 | 406 |
| 135 | 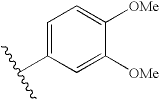 | 3 | 484 |

TABLE F

| | |
|---|---|
| | F |
| 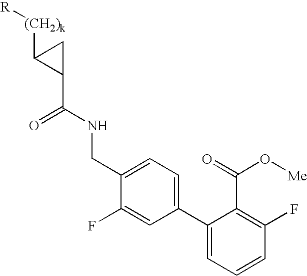 | |

| | | | LRMS |
|---|---|---|---|
| Example | R | k | (M + H⁺) |
| 136 | 4-pyridinyl | 2 | 451 |
| 137 | 2-pyridinyl | 2 | 451 |
| 138 | 3-pyridinyl | 2 | 451 |
| 139 | 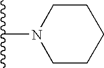 | 2 | 457 |
| 140 | 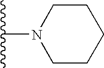 | 3 | 471 |

TABLE G

| | |
|---|---|
| | G |
| 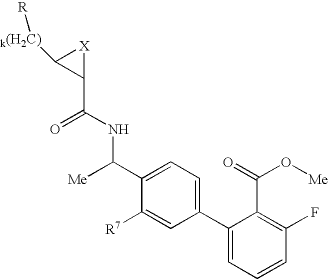 | |

| | | | | | | LRMS |
|---|---|---|---|---|---|---|
| Example | R | R7 | X | K | * | (M + H⁺) |
| 141 | 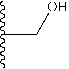 | H | CH₂ | 2 | R | 372 |
| 142 | 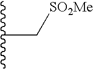 | H | CH₂ | 2 | R | 434 |
| 143 | 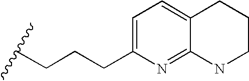 | F | H, H | 2 | R | 495 |
| 144 | 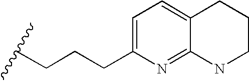 | F | H, H | 2 | S | 494 |

What is claimed is:

1. A compound of Formula (Ia), (Ib) or (Ic): 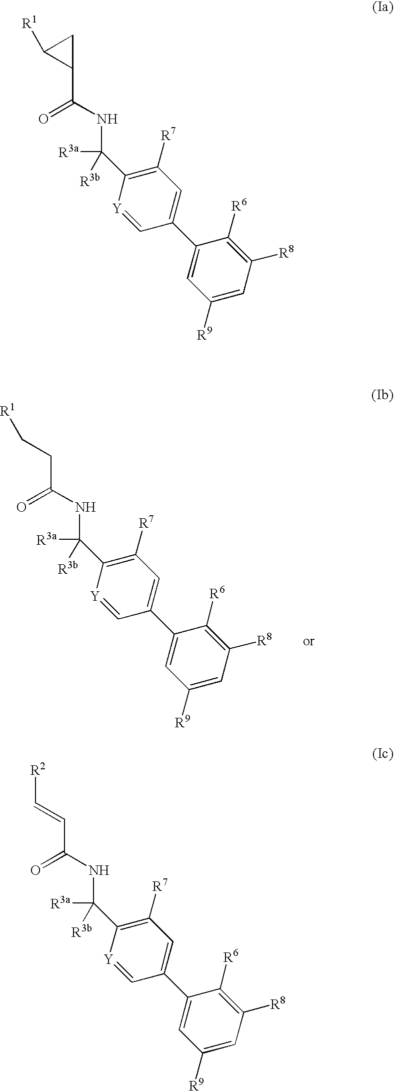
or a pharmaceutically acceptable salt thereof, wherein
k is 0, 1, 2, 3, 4, or 5;
m is 2, 3, or 4;
n is 1, 2 or 3;
p is 1, 2, 3, 4, or 5;
Y is CH or N;
R¹ is selected from —(CH₂)ₖR²⁰, —(CH₂)ₖR³⁰, —(CH₂)ₖR⁴⁰, —(CH₂)ₖR⁵⁰, and —(CH₂)ₙC(O)R³²;
R² is selected from —(CH₂)ₚR²⁰, —(CH₂)ₚR³⁰, —(CH₂)ₚR⁴⁰, —(CH₂)ₚR⁵⁰, and —(CH₂)ₚC(O)R³²;
R^{3a} and R^{3b} are independently selected from hydrogen, and C₁₋₄ alkyl optionally substituted with 1 to 5 halogen atoms;
R⁶ is selected from halogen, CF₃, CO₂R^{a}, C(O)NR^{b}R^{c}, OR^{a}, OSO₂R^{d}, and optionally substituted heterocycle where the heterocycle is a 5-membered heteroaromatic ring having a ring heteroatom selected from N, O and S, and optionally having up to 3 additional ring nitrogen atoms, 4,5-dihydro-oxazolyl and 4,5-dihydro-1,2,4-oxadiazolyl, and wherein said substituent is 1 to 3 groups independently selected from C₁₋₄ alkyl optionally substituted with 1 to 5 halogen atoms, OR^{a} or OC(O)R^{a};
R⁷ is selected from hydrogen and halogen;
R⁸ and R⁹ are independently selected from hydrogen, halogen, and C₁₋₄ alkyl optionally substituted with 1 to 5 halogen atoms;
R¹¹ is selected from (1) hydrogen, (2) (CH₂)ₖ-Ar optionally substituted with 1 to 3 groups independently selected from halogen, nitro, cyano, OR^{a}SR^{a}, CO₂R^{a}, C₁₋₄ alkyl and C₁₋₃ haloalkyl, wherein the Ar is selected from phenyl, pyridyl, 1,2-benzisothiazolyl, isoquinolinyl, 1,3-benzoxazolyl, quinazolinyl, 1,3-thiazolyl and 1,3,4-thiadiazolyl, (3) C(O)OR^{a}, (4) C(O)-Ar1, wherein Ar1 is selected from indolyl and 1,2,4-triazolyl, and (5) SO₂R^{d};
R¹² is 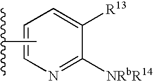
R¹³ and R¹⁴ are independently selected from hydrogen, C(O)OR^{a}, and C₁₋₄ alkyl optionally substituted with 1 to 5 groups independently selected from halogen, nitro, cyano and phenyl, or
R¹³ and R¹⁴ together form a bridging alkyl group of formula: (CH₂)ₘ;
R²⁰ is selected from (1) OR^{a}, (2) —O-phenyl optionally substituted with with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, (CH₂)ₖR²¹, (CH₂)ₖN(R^{a})OR^{a} and 4,5-dihydro-1H-imidazolyl, and (3) OC(O)NR^{b}R^{c};
R²¹ is selected from 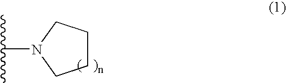
optionally substituted with 1 to 2 groups, not on the same carbon, independently selected from C₁₋₄ alkyl, C₁₋₄ hydroxyalkyl, OR^{a}, CO₂R^{a}, R^{a}, (CH₂)ₙOR^{a}, phenyl, CH₂NR^{b}R^{c}; and C(O)NR^{b}R^{c}; with the proviso that OR^{a} not be attached to a carbon attached to a nitrogen, 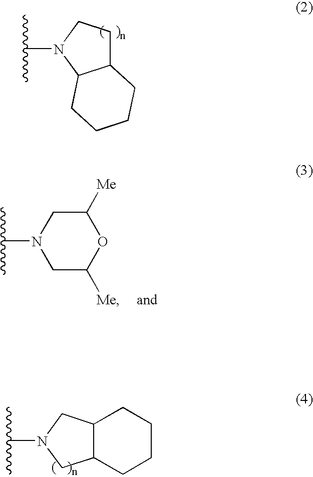
R³⁰ is selected from 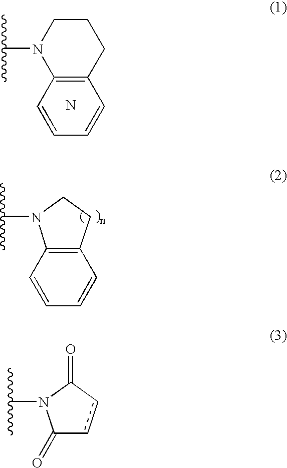
optionally benzofused,
(4) indolyl,
(5) NR^{b}R^{c},
(6) NR^{b}C(O)OR^{a},
(7) NR^{b}SO₂R^{d},
(8) NR^{b}C(O)R^{a},
(9) NR^{b}C(O)-pyrimidinyl,
(10) NR^{b}(CH₂)ₙR³³,
(11) NR^{3 1}(CH₂)ₖ-Ar2, wherein Ar2 is selected from phenyl,pyridyl and piperidinyl and Ar2 is optionally substituted with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl,
(12) NR^{b}(CH₂)ₖ-R¹²,
(13) N³⁰ (O—)R^{b}R^{c}, and
(14) a group selected from R³²;
R³¹ is selected from C(O)R^{a}, R^{a}, and SO₂R^{d};
R³² is selected from 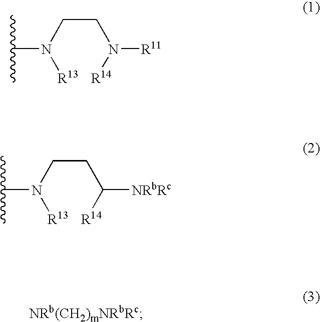
R³³ is 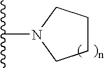
optionally substituted with 1 to 2 groups, not on the same carbon, independently selected from C₁₋₄ alkyl, C₁₋₄ haloalkyl, OR^{a}, CO₂R^{a}, R^{a}, (CH₂)ₙOR^{a}, phenyl, CH₂NR^{b}R^{c}; and C(O)NR^{b}R^{c}; with the proviso that OR^{a} not be attached to a carbon attached to a nitrogen;
R⁴⁰ is selected from SO₂(OR^{a}), SO₂R³², SO₂NR^{b}R^{c}, SO₂R^{d}, and SR^{a};
R⁵⁰ is selected from
(1) a group from R¹²,
(2) C(O)OR^{a},
(3) Hydroxyl,
(4) —S(O)₂—C₁₋₄ alkyl,
(5) Ar3 is selected from from phenyl, pyridyl, piperidinyl, naphthyridinyl, imidazolyl, benzimidazolyl, indolyl, and thiophenyl and Ar3 is optionally substituted with with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl;
R^{a} is selected from
(1) hydrogen,
(2) C₁₋₄ alkyl optionally substituted with 1 to 5 halogen atoms,
(3) (CH₂)ₖ-phenyl optionally substituted with 1 to 3 groups independently selected from halogen, cyano, nitro, OH, C₁₋₄ alkyloxy, C₃₋₆ cycloalkyl and C₁₋₄ alkyl optionally substituted with 1 to 5 halogen atoms,
(4) C₃₋₆ cycloalkyl, and
(5) pyridyl;
R^{b} and R^{c} are independently selected from
(1) hydrogen,
(2) C₁₋₄ alkyl optionally substituted with 1 to 5 groups independently selected from halogen, amino, mono-C₁₋₄alkylamino, di-C₁₋₄alkylamino, and SO₂R^{d},
(3) (CH₂)ₖ-phenyl optionally substituted with 1 to 3 groups selected from halogen, cyano, nitro, OH, C₁₋₄ alkyloxy, C₃₋₆ cycloalkyl and C₁₋₄ alkyl optionally substituted with 1 to 5 halogen atoms, and
(4) C₃₋₆ cycloalkyl, or
R^{b} and R^{c} together with the nitrogen atom to which they are attached form a 4-, 5-, or 6-membered aromatic or non-aromatic ring optionally containing an additional heteroatom selected from N, O, and S, wherein the S is optionally oxidized to the sulfone or sulfoxide; or
R^{b} and R^{c} together with the nitrogen atom to which they are attached form a cyclic imide;
R^{d} is selected from
(1) C₁₋₄ alkyl optionally substituted with 1 to 5 halogen atoms,
(2) C₁₋₄ alkyloxy,
(3) phenyl optionally substituted with 1 to 3 groups selected from halogen, cyano, nitro, OH, C₁₋₄ alkyloxy, C₃₋₆ cycloalkyl and C₁₋₄ alkyl optionally substituted with 1 to 5 halogen atoms, and
(4) pyridyl.

2. A compound according to claim 1 wherein
R¹ is selected from—(CH₂)ₖR²⁰, —(CH₂)ₖR³⁰, —(CH₂)ₖR⁴⁰, and—(CH₂)ₙC(O)R³²;
R² is selected from —(CH₂)ₚR²⁰, —(CH₂)ₚR⁴⁰, —(CH₂)ₚR⁵⁰, and—(CH₂)ₚC(O)R³²;
R^{3a} and R^{3b} are each independently selected from methyl and hydrogen;
R⁶ is selected from CF₃, CO₂R^{a}, C(O)NR^{b}R^{c}, OR^{a}, and optionally substituted heterocycle where the heterocycle is a 5-membered beteroaromatic ring having a ring heteroatom selected from N, O and S, and optionally having up to 3 additional ring nitrogen atoms, 4,5-dihydro-oxazolyl and 4,5-dihydro-1,2,4-oxadiazolyl, and wherein said substituent is 1 to 3 groups independently selected from C₁₋₄ alkyl optionally substituted with 1 to 5 halogen atoms, OR^{a} or OC(O)R^{a};
R⁷ is selected from fluorine and chlorine;
R⁹ is selected from hydrogen, fluorine and chlorine;
R⁸ is selected from fluorine and chlorine;
R¹¹ is selected from (1) (CH₂)ₖ-Ar optionally substituted with 1 to 3 groups independently selected from halogen, nitro, cyano, OR^{a}SR^{a}, CO₂R^{a}, C₁₋₄ alkyl and C₁₋₃ haloalkyl, wherein the Ar is selected from phenyl, pyridyl, 1,2-benzisothiazolyl, isoquinolinyl, 1,3-benzoxazolyl, quinazolinyl, 1,3-thiazolyl and 1,3,4-thiadiazolyl; (2) C(O)OR^{a}, and (3) SO₂R^{d};
R¹³ and R¹⁴ are independently selected from hydrogen and C(O)OR^{a}, or
R¹³ and R¹⁴ together form a bridging alkyl group of formula: (CH₂)ₘ;.
R²⁰ is selected from OR^{a}, and —O-phenyl optionally substituted with with 1 to 3 groups independently selected from halogen, OR^{a}, C₁₋₄ alkyl, C₁₋₃ haloalkyl, (CH₂)ₖNR^{b}R^{c}; and (CH₂)ₖR²¹;
R²¹ is 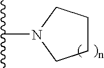
optionally substituted with 1 to 2 groups, not on the same carbon, independently selected from C₁₋₄ alkyl, C₁₋₄ hydroxyalkyl, OR^{a}, CO₂R^{a}, R^{a}, (CH₂)ₙOR^{a}, phenyl, CH₂NR^{b}R^{c}; and C(O)NR^{b}R^{c}; with the proviso that OR^{a} not be attached to a carbon attached to a nitrogen;
R³⁰ is selected from 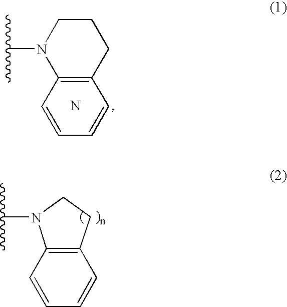
(3) NR^{b}R^{c},
(4) NR^{b}C(O)OR^{a},
(5) NR^{b}C(O)-pyrimidinyl,
(6) NR^{b}(CH₂)ₙR³³,
(7) NR³¹(CH₂)ₖ-Ar2, wherein Ar2 is selected from phenyl,pyridyl and piperidinyl and Ar2 is optionally substituted with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl,
(8) NR^{b}(CH₂)ₖ-R¹²,
(9) a group selected from R³²;
R³¹ is
R^{a};
R³³ is 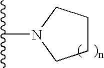
optionally substituted with 1 to 2 groups, not on the same carbon, independently selected from C₁₋₄ alkyl, OR^{a}, phenyl, and CH₂NR^{b}R^{c}; with the proviso that OR^{a} not be attached to a carbon attached to a nitrogen;
R⁴⁰ is selected from
(1) SO₂(OR^{a}), and
(2) SO₂R³²;
R⁵⁰ is selected from
(1) a group from R¹²,
(2) Hydroxyl, and
(3) Ar3 is selected from from phenyl, pyridyl, piperidinyl, naphthyridinyl, imidazolyl, benzimidazolyl, indolyl, and thiophenyl and Ar3 is optionally substituted with with 1 to 3 groups independently selected from halogen, nitro, cyano, OR^{a}, SR^{a}, C₁₋₄ alkyl, C₁₋₃ haloalkyl, CO₂R^{a}, (CH₂)ₖNR^{b}R^{c}, and 4,5-dihydro-1H-imidazolyl.
k is 0, 1, 2, or 4;
p is 0, 1, 2, 3 or 4;
m is 3 or 4; and
n is 1 or 2.

3. A compound according to claim 1 selected from
Methyl 3,3′-difluoro-4′-{[(3-hydroxybenzoyl)amino]methyl}biphenyl-2-carboxylate,
Methyl 3,3′-difluoro-4′-[({4-[(4-pyridin-4-ylpiperazin-1-yl)sulfonyl]benzoyl}amino)methyl]biphenyl-2-carboxylate,
Methyl 3,3′-difluoro-4′-{[(4-{[(2-piperidin-1-ylethyl)amino]sulfonyl}benzoyl)amino]methyl}biphenyl-2-carboxylate,
Methyl 3,3′-difluoro-4′-[({4-[(4-pyridin-4-ylpiperazin-1-yl)carbonyl]benzoyl}amino)methyl]biphenyl-2-carboxylate,
Methyl 4′-({[3-(aminomethyl)benzoyl]amino}methyl)-3,3′-difluorobiphenyl-2-carboxylate,
Methyl 3,3′-difluoro-4′-{[(3-{[(trifluoroacetyl)amino]methyl}benzoyl)amino]methyl}biphenyl-2-carboxylate,
Methyl 3,3′-difluoro-4′-{[(3-{[(3-piperidin-1-ylpropanoyl)amino]methyl}benzoyl)amino]methyl}biphenyl-2-carboxylate,
Methyl 3,3′-difluoro-4′-{[(3-{[(2-piperidin-1-ylethyl)amino]sulfonyl}benzoyl)amino]methyl}biphenyl-2-carboxylate,
Methyl 3,3′-difluoro-4′-[({3-[(pyrimidin-5-ylcarbonyl)amino]benzoyl}amino)methyl]biphenyl-2-carboxylate, and
Methyl 3,3′-difluoro-4′-({[2-(4-pyridin-4-ylpiperazin-1-yl)isonicotinoyl]amino}methyl)biphenyl-2-carboxylate.

4. A compound according to claim 1 of the Formula

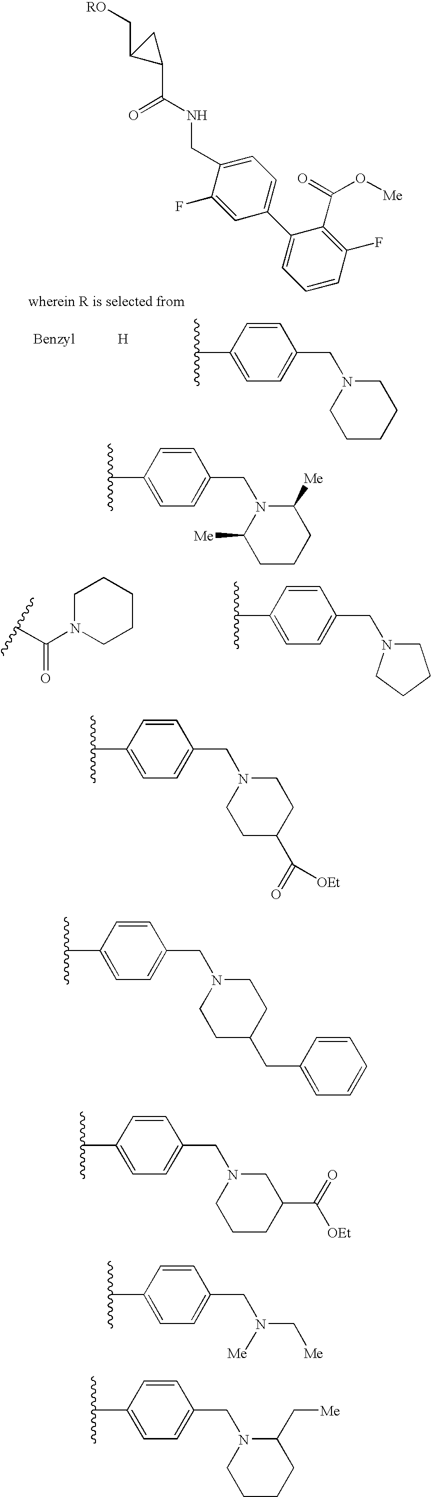

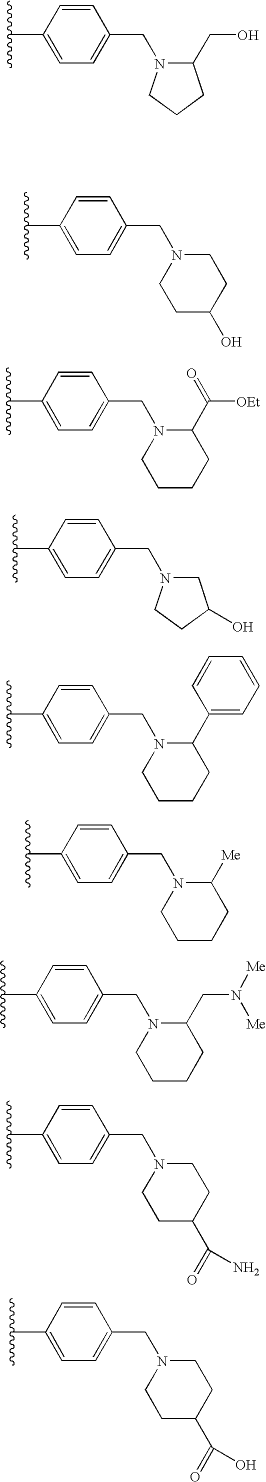

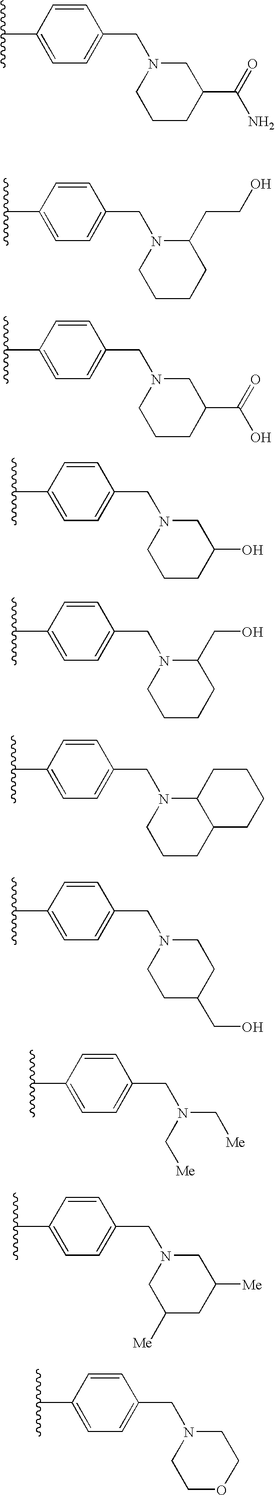

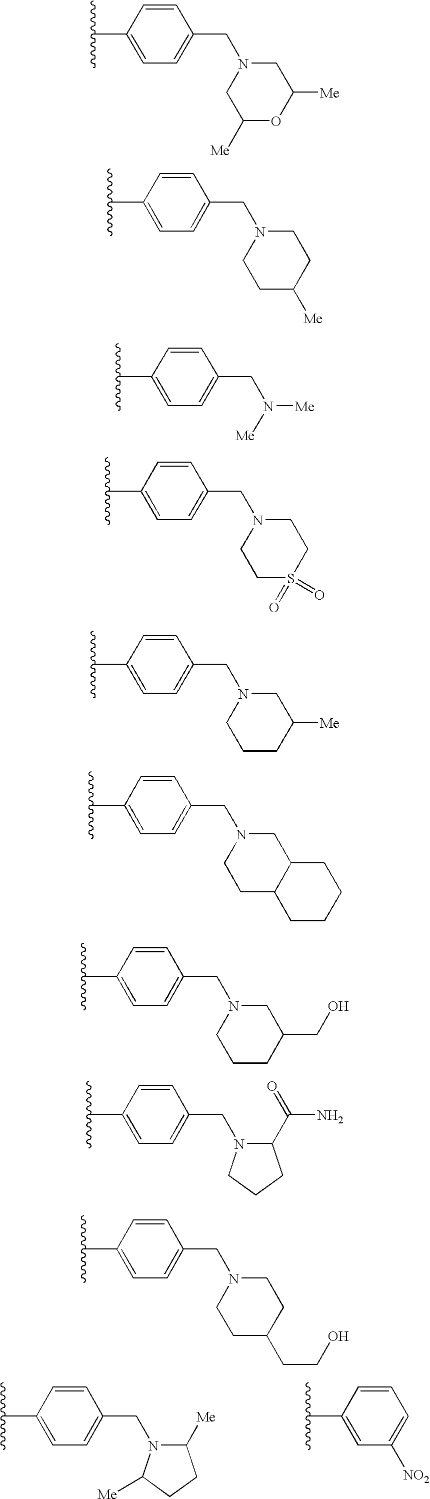

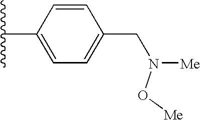

5. A compound according to claim 1 of the Formula

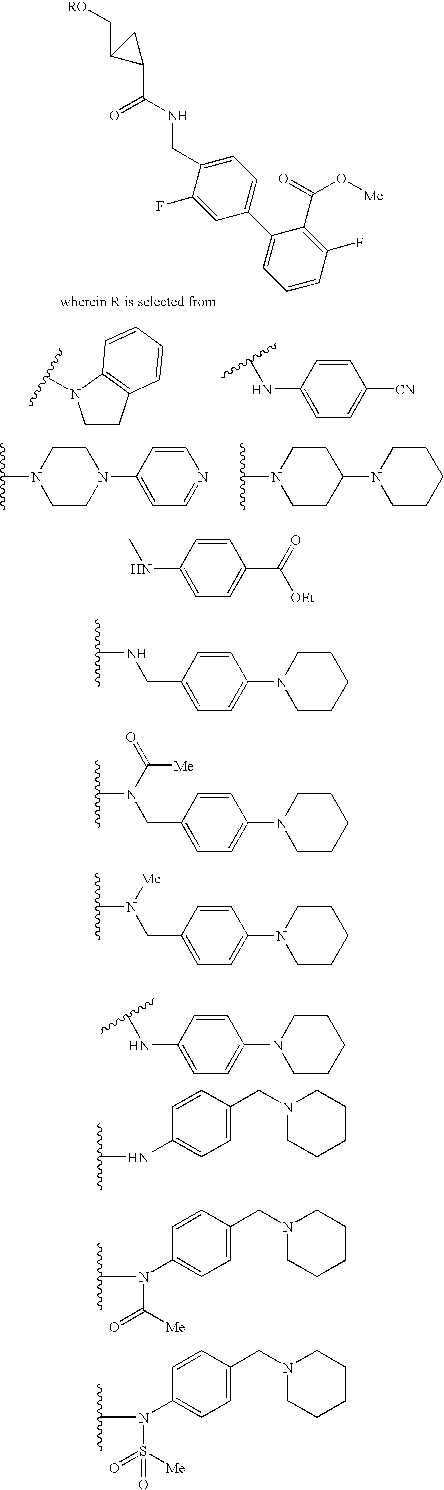

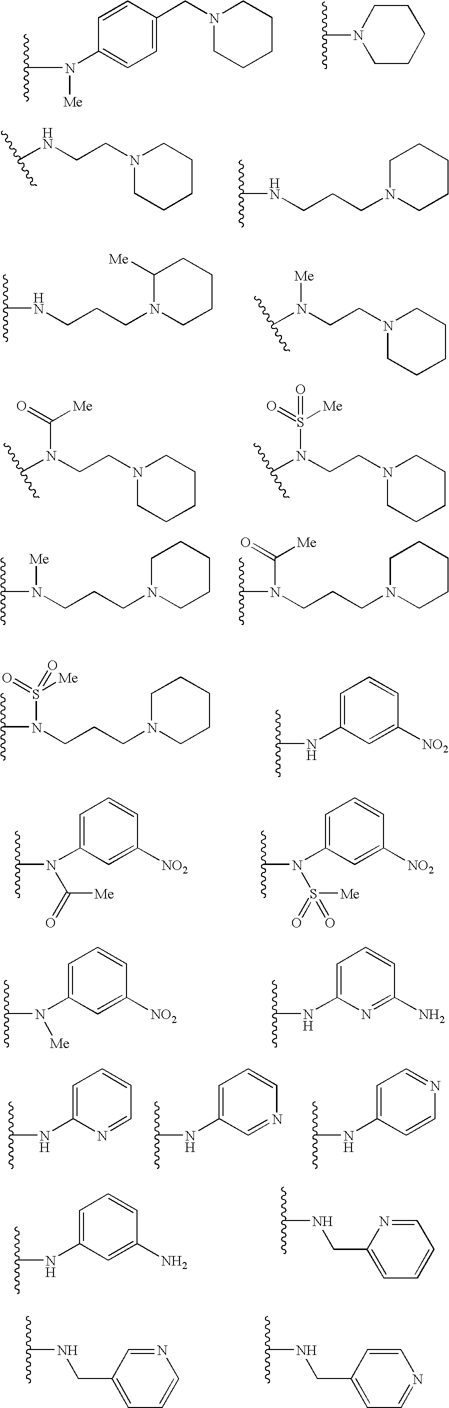

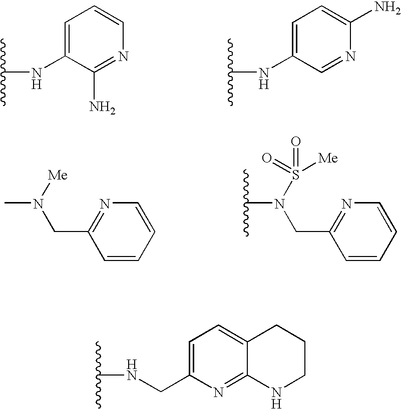

6. A compound according to claim 1 of the Formula 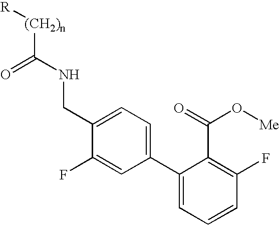
wherein

| R | n |
|---|---|
| 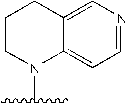 | 3 |
| 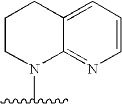 | 3 |
| 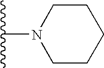 | 3 |
| 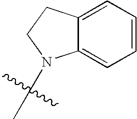 | 2 |
| 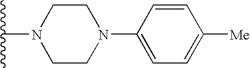 | 5 |
| 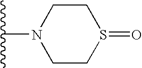 | 3 |
| 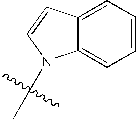 | 3 |
| 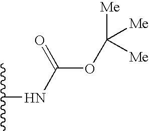 | 4 |
| 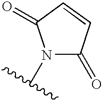 | 3 |
| 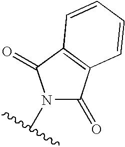 | 3 |
| 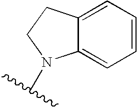 | 3 |
| 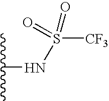 | 3 |
| 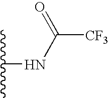 | 3 |
| 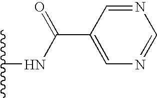 | 3 |
| 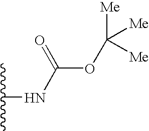 | 3 |
| SO₃H | 3 |
| SO₃H | 2 |
| 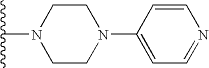 | 3 |
| 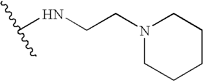 | 3 |
| SO₂NH₂ | 3 |

7. A compound according to claim 1 of the Formula

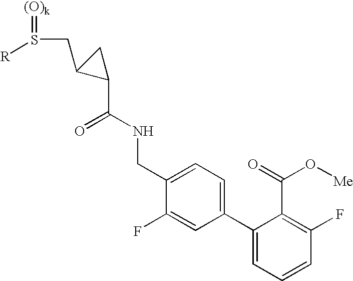

| R | k |
|---|---|
| Me | 0 |
| Et | 0 |
| 2-propyl | 0 |
| Phenyl | 0 |
| 2-pyridinyl | 0 |
| 4-pyridinyl | 0 |
| Et | 2 |
| 2-propyl | 2 |
| Phenyl | 2 |
| 2-pyridinyl | 2 |
| 4-pyridinyl | 2 |
| 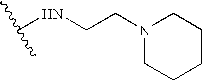 | 2 |
| 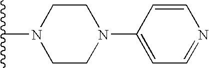 | 2 |

8. A compound according to claim 1 of the Formula 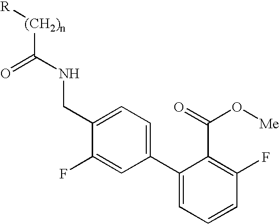
wherein

| R | n |
|---|---|
| 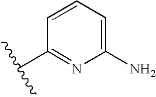 | 3 |
| 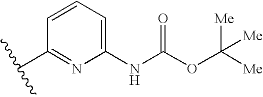 | 3 |
| 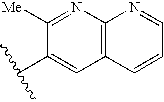 | 2 |
| 2-pyridinyl | 2 |
| 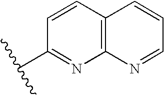 | 3 |
| 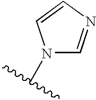 | 7 |
| 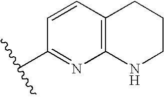 | 2 |
| 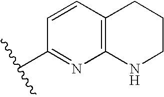 | 4 |
| 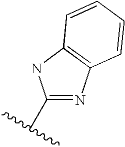 | 4 |
| 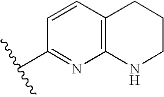 | 3 |
| 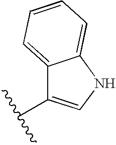 | 3 |
| 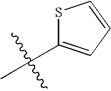 | 3 |
| CO₂Me | 3 |
| 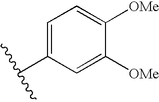 | 3 |

9. A compound according to claim 1 of Formula 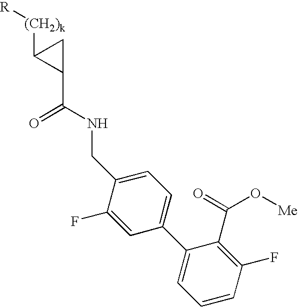
wherein

| Example | R | k |
|---|---|---|
| 136 | 4-pyridinyl | 2 |
| 137 | 2-pyridinyl | 2 |
| 138 | 3-pyridinyl | 2 |
| 139 | 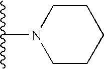 | 2 |
| 140 | 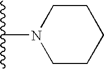 | 3 |

10. A compound according to claim 1 of Formula 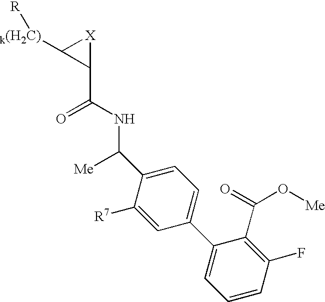
wherein

| R | R7 | X | k | * |
|---|---|---|---|---|
| 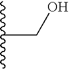 | H | CH₂ | 2 | R |
| 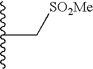 | H | CH₂ | 2 | R |
| 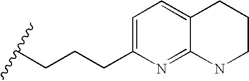 | F | H, H | 2 | R |
| 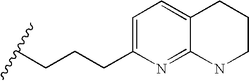 | F | H, H | 2 | S |

11. A pharmaceutical composition comprising a compound according to claim 1 or a pharmaceutically acceptable salt thereof; and a pharmaceutically acceptable carrier.

12. A method of treatment or prevention of pain and inflammation comprising a step of administering, to a subject in need of such treatment or prevention, an effective amount of a compound according to claim 1 or a pharmaceutically acceptable salt thereof.
